(19) United States
(12) Patent Application Publication
(10) Pub. No.: US 2017/0080161 A1
(43) Pub. Date: Mar. 23, 2017

(54) Drug Delivery Device

(71) Applicant: Sanofi-Aventis Deutschland GmbH, Frankfurt am Main (DE)

(72) Inventors: Matthew Meredith Jones, Warwickshire (GB); Samuel Keir Steel, Warwickshire (GB); Neal Alexander Blundred, South Coventry West Midlands (GB); Joseph Butler, Rugby Warwickshire (GB); Robert Frederick Veasey, Warwickshire (GB); David Plumptre, Worcestershire (GB)

(21) Appl. No.: 15/311,976

(22) PCT Filed: May 27, 2015
(86) PCT No.: PCT/EP2015/061635

§ 371 Date: Nov. 17, 2016

(30) Foreign Application Priority Data
May 28, 2014 (EP) 14170376.9

(51) Int. Cl. A61M 5/315; A61M 5/19

(52) U.S. Cl. CPC A61M 5/31536; A61M 5/19; A61M 5/31528; A61M 5/3155

(57) ABSTRACT

The invention is directed to a drug delivery device (1) comprising a primary drug delivery assembly (3) for the delivery of a primary medicament with a primary dose setting mechanism (8) and a secondary drug delivery assembly (4) for the delivery of a secondary medicament. A selection element (13) is movable in a first and a second direction and configured such that movement of the selection element (13) in the first direction and in the second direction causes the setting of an increasing dose in the primary dose setting mechanism (8).

The present invention is directed to a drug delivery device with a primary drug delivery assembly and a secondary drug delivery assembly.

Certain disease states require treatment using one or more different medicaments. Some drug compounds need to be delivered in a specific relationship with each other in order to deliver the optimum therapeutic dose. Here, combination therapy may be desirable, but not possible in a single formulation for reasons such as, but not limited to, stability, compromised therapeutic performance and toxicology.

For example, in some cases it might be beneficial to treat a diabetic with a long acting insulin and with a glucagon-like peptide-1 (GLP-1), which is derived from the transcription product of the proglucagon gene. GLP-1 is found in the body and is secreted by the intestinal L cell as a gut hormone. GLP-1 possesses several physiological properties that make it (and its analogs) a subject of intensive investigation as a potential treatment of diabetes mellitus. Another example of a medicament combination is the administration of a pain reliever in combination with a medicament for treating osteoarthritis.

Drug delivery devices of the aforementioned kind often have applications where regular injection by persons without formal medical training occurs. This is increasingly common among patients having diabetes or the like, e.g. osteoarthritis. Self-treatment enables such patients to conduct effective management of their disease.

In combination therapy, a primary medicament and a secondary medicament are delivered in a specific relationship to deliver the optimum therapeutic dose. The injection devices of the generic kind usually comprise a housing in which two or more drug delivery assemblies are retained. Such devices include a primary drug delivery assembly for dispensing the primary medicament such as the long-acting insulin and a secondary drug delivery assembly for dispensing the secondary medicament, such as GLP-1. Some kinds of drug delivery assemblies comprise a compartment such as a cartridge holder for accommodating a replaceable medicament container such as a cartridge which stores the medicament.

In some cases, depending on the patient or the stage of the therapy, an effective treatment requires variations in the quantities and/or proportions of the medicaments making up the combined therapy. For example, the patient may require a non-adjustable fixed dose of the secondary medicament in combination with an adjustable variable dose of the primary medicament.

The effectiveness of a combined delivery of medicaments may require one or more doses to be delivered sequentially with one of the two medicaments being injected into the human body prior to the delivery of the other medicament. Such treatment may be conducted with devices that include two separate dispensing mechanisms that are actuated independently from each other such that the dispensing mechanisms are activated successively. Sometimes patients require merely a dose of one of the medicaments, e.g. the primary medicament. The correct use of the device, however, may be hazardous for patients that are physically or mentally impaired or otherwise disadvantaged. It is desirable to have a device that is simple in terms of use and that has a high degree of user safety.

It is an object of the invention to improve the setting capabilities of a drug delivery device of the aforementioned kind and to provide a high degree of user convenience and safety in term of operating errors. In particular, it is desired to enable the user to choose either to dispense a dose of only one the medicaments or a combined dose of both medicaments.

The above problem is solved by a drug delivery device as defined in claim 1.

Drug delivery devices of the aforementioned kind comprise a primary drug delivery assembly for the delivery of a primary medicament with a primary dose setting mechanism to set a dose of a primary medicament contained in the primary drug delivery assembly and a secondary delivery assembly for the delivery of a secondary medicament. According to the invention, a selection element is provided which is movable in a first and a second direction and which is configured such that movement of the selection element in the first direction and in the second direction causes the setting of an increasing dose in the primary dose setting mechanism.

Preferably, two dose units are set in the primary drug delivery assembly, when the setting element is actuated.

The primary dose setting mechanism may also be configured as a primary dose setting and dose dispensing mechanism. The secondary drug delivery assembly may also include a secondary dose setting mechanism which may also be configured as a secondary dose setting and dose dispensing mechanism. The terms primary and secondary are substantially used for associating components with the respective drug delivery assembly.

The present invention provides for an effective mechanism to restrain the permitted user actions so that misadjustments during dose setting which may have a critical influence on the injection are prevented. Independent from the direction, in which the user moves the selection element, a positive dose in the primary drug delivery assembly is set. This efficiently restricts the degree of freedom the user has for setting doses in the combined drug delivery device. When the user actuates the selection element, a dose in the primary drug delivery assembly is always set. The present invention thereby enables for a precisely controlled combined therapy. At the same time, the technical framework is set for the user to choose between a single medication or a combined medication.

The drug delivery device may comprise a drug delivery device housing that extends from a proximal end to a distal end along a longitudinal axis and that accommodates the primary and the secondary drug delivery assembly. The distal end is usually referred to as the dispensing end where the drug delivery device may be equipped with a single dispense interface, such as a needle hub with an injection needle. The proximal end may be referred to as the setting end where a user operates a dose dial grip or the like to set medicament doses.

The housing may comprise a primary drug delivery assembly housing and may comprise a secondary drug delivery assembly housing that may be preferably releaseably attached to each other such that the single parts are replaceable.

The primary and the secondary drug delivery assembly housing may be configured to be attached to a medicament cartridge or a cartridge housing respectively. The cartridges may each contain a medicament reservoir. A primary medicament, e.g. a long-acting insulin, may be contained in primary reservoir that is configured to be coupled to the primary drug delivery assembly housing and a secondary medicament, e.g. GLP-1, may be contained in a secondary reservoir that is configured to be coupled to the secondary drug delivery assembly housing. Accordingly, two cartridges can be coupled to the drug delivery device.

The selection element may be formed as a selection switch or selection lever. The selection element may also be rotatable in both directions, preferably around a longitudinal axis that runs in axial direction of the secondary drug delivery assembly. The components of the secondary dose setting and also of the secondary dose dispensing mechanism may be substantially concentrically arranged with respect to said axis.

According to a further embodiment of the invention, the selection element is movable between a first, a second and a third position, wherein the selection element is movable from the first position into the second position in the first direction and from the first position into the third position in the second direction. The first direction may be different from the second direction. Preferably, the first direction is opposite the second direction. The selection element may be rotated in counterclockwise direction from the first into the second position and in clockwise direction from the first into the third position of the device when looking from the proximal end of the device to the distal end of the device.

The first position may be an intermediate or central position between the second and the third position and may constitute an “At Rest” state. This may correspond to a state prior to any setting actions the user perfoms to prepare the drug delivery device for later injection.

Preferably, the selection element is a dose setting element of the secondary drug delivery assembly. The selection element may be connected to the secondary drug delivery assembly, in particular a secondary dose setting mechanism of the secondary drug delivery assembly, such that actuation of the selection element is transferred to the secondary dose setting mechanism and sets a dose of a secondary medicament in the secondary drug delivery assembly.

According to a further embodiment of the invention, the selection element is connected to secondary dose setting mechanism such that movement of the selection element in the first direction, resp. from the first position into the second position sets a preferably fixed or predetermined dose in the secondary dose setting mechanism.

Preferably, the primary drug delivery assembly is configured for the setting of a variable dose of a primary medicament to be dispensed by means of the primary drug delivery assembly and the secondary drug delivery assembly is configured for the setting of a fixed dose of a secondary medicament to be dispensed by means of the secondary drug delivery assembly. Accordingly, the primary drug delivery assembly may comprise a variable dose setting mechanism and the secondary drug delivery assembly may comprise a fixed dose setting mechanism. Each of the mechanisms may be configured for dispense, also.

The term “fixed dose” as used herein can be characterized as a dose value that is defined by the construction of the drug delivery assembly or the drug delivery device, wherein the user is only able to inject a specific dose. The user is not in the position to set lower or higher doses of medicament and/or to inject lower or higher doses of the medicament. The dose the user may effectively set and inject is restricted to a certain value. Accordingly, the setting element may be used to set a fixed dose of a secondary medicament to be dispensed with the secondary drug delivery assembly.

On the contrary, the term “variable dose” can be characterized as a dose where the user is substantially free to choose the amount of medicament he wants to inject. The dose is variably adjustable, normally between upper and lower limits.

According to a further embodiment of the invention, movement of the selection element in the second direction, resp. from the first position into the third position, prevents the secondary dose setting mechanism from setting a dose or does not set a dose in the secondary drug delivery assembly. This may be achieved by a ratchet mechanism in the secondary dose setting mechanism that prevents the transmission of rotational movement of the setting element to the secondary dose setting mechanism. With the selection element, the user can easily chose to only set a dose of the primary medicament in the primary drug delivery assembly or to set a dose of the primary medicament in combination with a dose of the secondary medicament.

According to a further embodiment of the invention, the primary drug delivery assembly comprises a primary dose dial sleeve configured to rotate during dose setting. The selection element may be configured to be connected to the primary dose dial sleeve such that movement of the selection element from the first position into the second and/or the third position causes rotation of the primary dose dial sleeve thereby dialing an increasing dose in the primary dose setting mechanism.

According to a further embodiment, the primary drug delivery assembly comprises a biasing member, such as a spring, configured to be prestressed during dose setting of the primary drug delivery assembly and to be relaxed during dose dispense such as to drive a primary lead screw in distal direction. Thereby, the mechanical energy for later dispense of a primary medicament may be established during the setting of a dose of the secondary medicament.

To efficentily constrain and to regulate the sequence of user action, a further embodiment of the invention includes a lock mechanism that is configured to lock a primary dose dispensing mechanism, respectively a primary dose setting and dispensing mechanism of the primary drug delivery assembly against dispensing when the selection element is actuated or moved by the user. The lock mechanism may lock the primary dose dispensing mechanism or at least elements of the mechanism against movement such that movement of a primary lead screw in distal direction is prevented and a primary medicament cannot be dispensed.

It has been proven effective when the lock mechanism engages the primary dose dispensing mechanism when a dose in the secondary dose setting mechanism is set. The lock mechanism may be coupled to the setting element such that the lock mechanism is moved or switched or set from an unlocked state, in which dispense of a primary medicament is allowed, into a locked state, in which dispense of the primary medicament is prevented, when the setting element is moved from the first into the second position, respectively when a dose is set in the secondary drug delivery assembly.

By providing a lock mechanism, the primary dose dispensing mechanism may be prevented from dispensing before the second dose dispensing mechanism has finished dispensing. Via this interlock it is ensured, that e.g. a fixed dose set in the secondary drug delivery assembly is dispensed prior to a variable dose set in the primary drug delivery assembly. Such interlock feature efficiently prevents operating errors by the user so that a clear procedure during dose setting must be followed.

According to a further embodiment, the lock mechanism comprises a ratchet and a clutch, wherein in the locked state, the clutch is rotationally locked to the ratchet. In the unlocked state, the clutch is not rotationally locked to the ratchet. In the locked state, the ratchet may engage the clutch such that rotation of the clutch is prevented. In the unlocked state, the ratchet may be disengaged from the clutch.

The ratchet may be rotationally constrained with respect to a longitudinal axis of the secondary drug delivery assembly but axially moveable in axial direction or with respect to the drug delivery device housing. This may be achieved by a splined interface with the drug delivery device housing that extends in axial direction. The ratchet may be moveable between a proximal and a distal position and may engage the clutch when moved from the proximal into the distal position.

According to a further embodiment, the ratchet is configured to engage the clutch when a dose in the secondary drug delivery assembly is set. The ratchet may be connected to the setting element such that movement of the setting element from the first into the second position moves the ratchet into engagement with the clutch. Thereby, the axial position of the ratchet may be dependent upon the state of the fixed dose setting mechanism.

The primary dose dispensing mechanism may comprise a primary lead screw, a piston rod or the like configured to move in distal direction during dose dispensing. The clutch may be rotationally constrained to the primary lead screw of the primary drug delivery device. The primary lead screw may be configured to rotate during dose dispense. The primary lead screw may be in threaded engagement with the primary drug delivery assembly housing such that rotation of the primary lead screw causes distal movement of the primary lead screw. The primary lead screw may be rotationally constrained to the clutch via a splined interface. By locking the clutch against rotation, the primary lead screw may be prevented from rotating and from advancing in distal direction.

According to a further embodiment, the secondary drug delivery assembly comprises a secondary drive sleeve configured to move distally during dose setting such that the ratchet engages the clutch. Preferably, the secondary drive sleeve moves distally in a pure axial motion during dose setting and drives the ratchet into engagement with the clutch.

The secondary drive sleeve may be connected to the selection element via a coupling mechanism which is configured such that rotational movement of the selection element in the first direction is transferred into a preferably pure axial motion of the secondary drive sleeve in distal direction. The coupling mechanism may also be configured such that rotation of the setting element in the second direction causes the secondary drive sleeve to maintain its axial position.

According to a further embodiment, the secondary drug delivery assembly comprises a biasing element such as a spring configured to bias the ratchet in proximal direction. The biasing element may be positioned distally from the ratchet. Thereby, energy for disengaging the ratchet from the clutch and for disabling the interlock mechanism may be stored.

According to a further embodiment, the biasing element is arranged such that movement of the ratchet into engagement with the clutch compresses the biasing element. Preferably, distal movement of the secondary drive sleeve causes the ratchet to move distally and to engage the clutch and the biasing element to be compressed. This provides for a clear connection between the setting action in the secondary drug delivery assembly and the locked state of the lock mechanism. Further, the biasing element may store the mechanical energy for later dispense. Accordingly, the biasing element may be configured as a secondary dispensing spring.

The ratchet may be configured to disengage from the clutch during dose dispense of the secondary medicament and/or when the secondary dose setting mechanism is reset. The secondary drive sleeve may be configured to move in proximal direction during dose dispense of the secondary drug delivery assembly, so that the ratchet biases the secondary drive sleeve in proximal direction under the force of the relaxing biasing element.

The secondary drive sleeve may be configured to move in proximal direction during dose dispense of the secondary drug delivery assembly in a helical movement. Further, the secondary drive sleeve may be rotationally constrained to a secondary lead screw that is in threaded engagement with the secondary drug delivery assembly housing such that rotation of the drive sleeve is transferred to the secondary lead screw which is caused to move in distal direction. The secondary drive sleeve may interface with the secondary lead screw by way of a spline connection, wherein movement of the secondary drive sleeve in distal direction relative to the secondary lead screw corresponds to the setting of a dose in the secondary drug delivery assembly.

A helical movement may be defined as a translational movement in axial direction combined with a rotation around the axis of the translational movement.

According to a further embodiment of the invention, the secondary drug delivery assembly comprises a secondary inner body configured to transfer axial movement of the secondary drive sleeve in proximal direction into a helical motion under the force of the secondary dispensing spring. To enable for axial movement of the secondary drive sleeve during dose setting, the secondary drive sleeve may interface with the secondary inner body via a set of mating axial ratchet teeth such that the secondary drive sleeve is prevented from rotating during dose setting when the axial ratchet teeth are fully engaged.

The drug delivery device may comprise an actuation element such as a trigger, preferably movable in axial direction to initiate the dispense of the drug delivery device. A lens or any other suitable movable element may be moved in proximal direction during the setting of a dose in the secondary drug delivery assembly and may be moved in distal direction when the trigger is actuated. Said distal movement may be transferred to the secondary drive sleeve such that the dispense in the secondary drug delivery assembly is initiated.

The coupling mechanism may comprise a selection sleeve and a setting sleeve both configured to engage each other through a ratchet mechanism. The ratchet mechanism may be configured such that rotation of the selection sleeve in a first direction is transferred to the setting sleeve and such that rotation of the selection sleeve in a second direction causes the selection sleeve to rotate relative to the setting sleeve. The selection sleeve may be rotationally constrained to a selection shaft and may be axially movable with respect to the selection shaft. The selection sleeve may be provided with integrated sprung elements, which act to force the selection sleeve into engagement with the setting sleeve. The selection shaft may be axially constrained in the drug delivery device housing. Further, the selection shaft may be rotationally constrained to the selection element.

The secondary inner body may provide a common principle axis for the secondary drug delivery assembly about which all other components of the secondary dose setting mechanism can be located concentrically. The setting sleeve may be free to move axially along the secondary inner body. Its rotation may be constrained by abutments with the secondary inner body.

The secondary drive sleeve may interface with the setting sleeve via a set of opposing axial ratchet teeth or slanted surfaces such that rotation of the setting sleeve in the first direction forces the drive sleeve to move axially towards the distal end of the device. Further, the secondary drive sleeve may engage the ratchet though a rotational one-way coupling, e.g. through mating axial ratchet teeth such that during dose setting, the secondary drive sleeve is prevented from rotating.

The setting sleeve may be configured to disengage from the secondary drive sleeve during dose dispense such that the secondary drive sleeve is free to move in proximal direction under the force of the biasing element such as to engage the secondary inner body and to rotate by engagement with the secondary inner body.

According to a further embodiment, a transfer element is provided which is configured to transfer movement of the selection element to the primary dose setting mechanism such that a dose in the primary drug delivery assembly is set. Preferably, the transfer element is movable between a first and a second position.

The transfer element may be a sliding element that is slidably guided in the drug delivery device housing and may be configured to engage the primary dose dial sleeve such that lateral motion of the transfer element causes rotation of the primary dose dial sleeve. The transfer element may be arranged such as to be shifted by the selection element into engagement with the primary dose dial sleeve such that the primary dose dial sleeve is rotated. The transfer element may be activated by abutment with the selection element.

The selection element may be provided with a profiled abutment which forces the transfer element into engagement with the primary dose dial sleeve wherein the abutment may be arranged on the setting element and/or on the selection shaft such that rotation of the setting element from the first into the second or from the first into the third position causes the transfer element to engage the primary dose dial sleeve such that the primary dose dial sleeve is rotated and a dose of a primary medicament to be dispensed is set in the primary drug delivery assembly. When the selection element is rotated from the first into the second position, rotation of the selection element is transferred to the primary dose dial sleeve and is also transferred to the secondary drive sleeve which is moved in distal direction.

By moving the selection element from the first into the second position, a predetermined dose of the primary medicament and a predetermined dose of the secondary medicament are set. On the contrary, when the selection switch is moved from the first into the third position, movement of the selection element is only transferred to the primary dose dial sleeve and a dose of the secondary medicament is not set. In each case, the selection movement of the selection element from the first into the second position or into the third position causes the setting of a dose in the primary drug delivery assembly.

This efficiently limits the possible setting actions a user can conduct to prepare his device for injection. It is efficiently prevented that the user sets a dose of a secondary medicament without a dose of the primary medicament. Further, the mechanism enables the user to turn off the fixed dose in the secondary drug delivery assembly and to “split” the dose. The automatic dialing of a predetermined unit of a primary medicament in the primary drug delivery assembly enables for an effective user selection regarding his choice whether he wants to inject merely a dose of the primary medicament or a combined dose of a primary and a secondary medicament.

The selection element and/or the drug delivery device housing may be provided with means to define the possible degree of movement, respectively rotation. For example, the possible degree of rotation may be defined by engagement between a selection element and/or the selection shaft and a counter abutment or the like on the drug delivery device housing. Thereby, the value of a fixed dose can be efficiently set.

According to a further embodiment, a further interlock mechanism is provided that functions as a latching mechanism and that is configured to lock the selection element and/or the selection shaft in the second and/or the third position. This mechanism may be an additional or alternative measure to efficiently put sequential delivery of medicaments into practice. Especially combined with the lock mechanism comprising the ratchet and the clutch, efficient measures are provided that control the sequence of the medicament delivery.

For this purpose, a latch may be provided that is axially movable with respect to the selection shaft between a distal and a proximal position, wherein the latch locks the selection shaft against rotation in the distal position. A biasing member urges the latch in distal direction against the primary dose dial sleeve, wherein rotation of the primary dose dial sleeve causes the latch to move to its distal position. The latch may be provided with a radial abutment configured to engage a profiled boss on the selection shaft when the latch is in the distal position such that the selection shaft is prevented from rotating back from the third or second position into the first position.

The primary dose dial sleeve may be provided with an abutment arranged on the dose dial sleeve such as to prevent distal movement of the latch when the primary dose dial sleeve is in a position that corresponds to a set dose of zero units. This mechanism provides an efficient interlock, which maintains the selection element in either of its rotated positions until the primary dose dial sleeve returns to the zero dose position.

According to a further embodiment of the invention, a further interlock mechanism is configured as a limiting means that limits the setting of a dose in the primary drug delivery assembly to a predetermined value when the selection element is in the first position and that allows the setting of higher doses when the selection element is in the second or third position.

The limiting mechanism may comprise a sliding element, e.g. a two units interlock slider. The selection shaft may be configured to force the sliding element into engagement with the primary dose dial sleeve when the setting element is in the first position. Preferably, the selection shaft is provided with a cam feature that is arranged on the selection shaft such that the interlock slider is prevented from disengaging from the primary dose dial sleeve when the selection element is in the first position. Accordingly, when the selection element is in the second and/or the third position, the interlock slider is free to disengage from the primary dose dial sleeve, preferably by rotation of the primary dose dial sleeve. The interlock slider may effectively limit the rotation of the primary dose dial sleeve to a predetermined number of units, e.g. two units which is why the sliding element may be referred as a two unit interlock slider that prevents further rotation of the primary dose dial sleeve after the primary dose dial sleeve has been rotated up to two units. This interlock mechanism effectively limits the selectable dose of the primary drug delivery assembly to two units which may equate to a prime dose. Unless the user choses to either set or not to set a fixed dose in the secondary drug delivery assembly by moving the selection element in the second or third position, a further dose in the primary drug delivery assembly cannot be set. By forcing the user to make a choice before using the device, they are efficiently encouraged to be conscious about the actions and the risk of a mistake is effectively reduced.

According to a further embodiment, the drug delivery device comprises a primary medicament cartridge coupled to the primary drug delivery assembly and a secondary medicament cartridge coupled to the secondary drug delivery assembly.

The fixed dose setting and dispensing mechanism and the variable dose setting and dispensing mechanism could be configured separable to allow a single expended cartridge from either the primary or the secondary drug delivery assembly to be disposed of and replaced. It is proposed that the secondary drug delivery assembly can be separated between the selection sleeve and the setting sleeve. Drug wastage can be efficiently minimized thereby.

The drug delivery device can be a disposable injection device. Such devices can be thrown away or recycled after the content of the medicaments has been exhausted. However, the present invention is also applicable with re-usable devices designed to replace an empty cartridge with a filled one after the whole content of the former cartridge has been administered.

The term “medicament”, as used herein, means a pharmaceutical formulation containing at least one pharmaceutically active compound,

wherein in one embodiment the pharmaceutically active compound has a molecular weight up to 1500 Da and/or is a peptide, a proteine, a polysaccharide, a vaccine, a DNA, a RNA, an enzyme, an antihousing or a fragment thereof, a hormone or an oligonucleotide, or a mixture of the above-mentioned pharmaceutically active compound,

wherein in a further embodiment the pharmaceutically active compound is useful for the treatment and/or prophylaxis of diabetes mellitus or complications associated with diabetes mellitus such as diabetic retinopathy, thromboembolism disorders such as deep vein or pulmonary thromboembolism, acute coronary syndrome (ACS), angina, myocardial infarction, cancer, macular degeneration, inflammation, hay fever, atherosclerosis and/or rheumatoid arthritis,

wherein in a further embodiment the pharmaceutically active compound comprises at least one peptide for the treatment and/or prophylaxis of diabetes mellitus or complications associated with diabetes mellitus such as diabetic retinopathy, wherein in a further embodiment the pharmaceutically active compound comprises at least one human insulin or a human insulin analogue or derivative, glucagon-like peptide (GLP-1) or an analogue or derivative thereof, or exendin-3 or exendin-4 or an analogue or derivative of exendin-3 or exendin-4.

Insulin analogues are for example Gly(A21), Arg(B31), Arg(B32) human insulin; Lys(B3), Glu(B29) human insulin; Lys(B28), Pro(B29) human insulin; Asp(B28) human insulin; human insulin, wherein proline in position B28 is replaced by Asp, Lys, Leu, Val or Ala and wherein in position B29 Lys may be replaced by Pro; Ala(B26) human insulin; Des(B28-B30) human insulin; Des(B27) human insulin and Des(B30) human insulin.

Insulin derivates are for example B29-N-myristoyl-des(B30) human insulin; B29-N-palmitoyl-des(B30) human insulin; B29-N-myristoyl human insulin; B29-N-palmitoyl human insulin; B28-N-myristoyl LysB28ProB29 human insulin; B28-N-palmitoyl-LysB28ProB29 human insulin; B30-N-myristoyl-ThrB29LysB30 human insulin; B30-N-palmitoyl-ThrB29LysB30 human insulin; B29-N-(N-palmitoyl-Y-glutamyl)-des(B30) human insulin; B29-N-(N-lithocholyl-Y-glutamyl)-des(B30) human insulin; B29-N-(ω-carboxyheptadecanoyl)-des(B30) human insulin and B29-N-(ω-carboxyhepta-decanoyl) human insulin.

Exendin-4 for example means Exendin-4(1-39), a peptide of the sequence H-His-Gly-Glu-Gly- Thr-Phe-Thr-Ser-Asp-Leu-Ser-Lys-Gln-Met-Glu-Glu-Glu-Ala-Val-Arg-Leu-Phe-Ile-Glu-Trp-Leu-Lys-Asn-Gly-Gly-Pro-Ser-Ser-Gly-Ala-Pro-Pro-Pro-Ser-NH2.

Exendin-4 derivatives are for example selected from the following list of compounds:

H-(Lys)4-des Pro36, des Pro37 Exendin-4(1-39)-NH2,

H-(Lys)5-des Pro36, des Pro37 Exendin-4(1-39)-NH2,

des Pro36 Exendin-4(1-39),

des Pro36 [Asp28] Exendin-4(1-39),

des Pro36 [IsoAsp28] Exendin-4(1-39),

des Pro36 [Met(O)14, Asp28] Exendin-4(1-39),

des Pro36 [Met(O)14, IsoAsp28] Exendin-4(1-39),

des Pro36 [Trp(O2)25, Asp28] Exendin-4(1-39),

des Pro36 [Trp(O2)25, IsoAsp28] Exendin-4(1-39),

des Pro36 [Met(O)14 Trp(O2)25, Asp28] Exendin-4(1-39),

des Pro36 [Met(O)14 Trp(O2)25, IsoAsp28] Exendin-4(1-39); or

des Pro36 [Asp28] Exendin-4(1-39),

des Pro36 [IsoAsp28] Exendin-4(1-39),

des Pro36 [Met(O)14, Asp28] Exendin-4(1-39),

des Pro36 [Met(O)14, IsoAsp28] Exendin-4(1-39),

des Pro36 [Trp(O2)25, Asp28] Exendin-4(1-39),

des Pro36 [Trp(O2)25, IsoAsp28] Exendin-4(1-39),

des Pro36 [Met(O)14 Trp(O2)25, Asp28] Exendin-4(1-39),

des Pro36 [Met(O)14 Trp(O2)25, IsoAsp28] Exendin-4(1-39),

wherein the group -Lys6-NH2 may be bound to the C-terminus of the Exendin-4 derivative;

or an Exendin-4 derivative of the sequence

des Pro36 Exendin-4(1-39)-Lys6-NH2 (AVE0010),

H-(Lys)6-des Pro36 [Asp28] Exendin-4(1-39)-Lys6-NH2,

des Asp28 Pro36, Pro37, Pro38Exendin-4(1-39)-NH2,

H-(Lys)6-des Pro36, Pro38 [Asp28] Exendin-4(1-39)-NH2,

H-Asn-(Glu)5des Pro36, Pro37, Pro38 [Asp28] Exendin-4(1-39)-NH2,

des Pro36, Pro37, Pro38 [Asp28] Exendin-4(1-39)-(Lys)6-NH2,

H-(Lys)6-des Pro36, Pro37, Pro38 [Asp28] Exendin-4(1-39)-(Lys)6-NH2,

H-Asn-(Glu)5-des Pro36, Pro37, Pro38 [Asp28] Exendin-4(1-39)-(Lys)6-NH2,

H-(Lys)6-des Pro36 [Trp(O2)25, Asp28] Exendin-4(1-39)-Lys6-NH2,

H-des Asp28 Pro36, Pro37, Pro38 [Trp(O2)25] Exendin-4(1-39)-NH2,

H-(Lys)6-des Pro36, Pro37, Pro38 [Trp(O2)25, Asp28] Exendin-4(1-39)-NH2,

H-Asn-(Glu)5-des Pro36, Pro37, Pro38 [Trp(O2)25, Asp28] Exendin-4(1-39)-NH2,

des Pro36, Pro37, Pro38 [Trp(O2)25, Asp28] Exendin-4(1-39)-(Lys)6-NH2,

H-(Lys)6-des Pro36, Pro37, Pro38 [Trp(O2)25, Asp28] Exendin-4(1-39)-(Lys)6-NH2,

H-Asn-(Glu)5-des Pro36, Pro37, Pro38 [Trp(O2)25, Asp28] Exendin-4(1-39)-(Lys)6-NH2,

H-(Lys)6-des Pro36 [Met(O)14, Asp28] Exendin-4(1-39)-Lys6-NH2,

des Met(O)14 Asp28 Pro36, Pro37, Pro38 Exendin-4(1-39)-NH2,

H-(Lys)6-desPro36, Pro37, Pro38 [Met(O)14, Asp28] Exendin-4(1-39)-NH2,

H-Asn-(Glu)5-des Pro36, Pro37, Pro38 [Met(O)14, Asp28] Exendin-4(1-39)-NH2,

des Pro36, Pro37, Pro38 [Met(O)14, Asp28] Exendin-4(1-39)-(Lys)6-NH2,

H-(Lys)6-des Pro36, Pro37, Pro38 [Met(O)14, Asp28] Exendin-4(1-39)-(Lys)6-NH2,

H-Asn-(Glu)5 des Pro36, Pro37, Pro38 [Met(O)14, Asp28] Exendin-4(1-39)-(Lys)6-NH2,

H-Lys6-des Pro36 [Met(O)14, Trp(O2)25, Asp28] Exendin-4(1-39)-Lys6-NH2,

H-des Asp28 Pro36, Pro37, Pro38 [Met(O)14, Trp(O2)25] Exendin-4(1-39)-NH2,

H-(Lys)6-des Pro36, Pro37, Pro38 [Met(O)14, Asp28] Exendin-4(1-39)-NH2,

H-Asn-(Glu)5-des Pro36, Pro37, Pro38 [Met(O)14, Trp(O2)25, Asp28] Exendin-4(1-39)-NH2,

des Pro36, Pro37, Pro38 [Met(O)14, Trp(O2)25, Asp28] Exendin-4(1-39)-(Lys)6-NH2,

H-(Lys)6-des Pro36, Pro37, Pro38 [Met(O)14, Trp(O2)25, Asp28] Exendin-4(1-39)-(Lys)6-NH2,

H-Asn-(Glu)5-des Pro36, Pro37, Pro38 [Met(O)14, Trp(O2)25, Asp28] Exendin-4(1-39)-(Lys)6-NH2;

or a pharmaceutically acceptable salt or solvate of any one of the afore-mentioned Exendin-4 derivative.

Hormones are for example hypophysis hormones or hypothalamus hormones or regulatory active peptides and their antagonists as listed in Rote Liste, ed. 2008, Chapter 50, such as Gonadotropine (Follitropin, Lutropin, Choriongonadotropin, Menotropin), Somatropine (Somatropin), Desmopressin, Terlipressin, Gonadorelin, Triptorelin, Leuprorelin, Buserelin, Nafarelin, Goserelin.

A polysaccharide is for example a glucosaminoglycane, a hyaluronic acid, a heparin, a low molecular weight heparin or an ultra low molecular weight heparin or a derivative thereof, or a sulphated, e.g. a poly-sulphated form of the above-mentioned polysaccharides, and/or a pharmaceutically acceptable salt thereof. An example of a pharmaceutically acceptable salt of a poly-sulphated low molecular weight heparin is enoxaparin sodium.

Antibodies are globular plasma proteins (˜150 kDa) that are also known as immunoglobulins which share a basic structure. As they have sugar chains added to amino acid residues, they are glycoproteins. The basic functional unit of each antihousing is an immunoglobulin (Ig) monomer (containing only one Ig unit); secreted antibodies can also be dimeric with two Ig units as with IgA, tetrameric with four Ig units like teleost fish IgM, or pentameric with five Ig units, like mammalian IgM.

The Ig monomer is a “Y”-shaped molecule that consists of four polypeptide chains; two identical heavy chains and two identical light chains connected by disulfide bonds between cysteine residues. Each heavy chain is about 440 amino acids long; each light chain is about 220 amino acids long. Heavy and light chains each contain intrachain disulfide bonds which stabilize their folding. Each chain is composed of structural domains called Ig domains. These domains contain about 70-110 amino acids and are classified into different categories (for example, variable or V, and constant or C) according to their size and function. They have a characteristic immunoglobulin fold in which two β sheets create a “sandwich” shape, held together by interactions between conserved cysteines and other charged amino acids.

There are five types of mammalian Ig heavy chain denoted by α, δ, ε, γ, and μ. The type of heavy chain present defines the isotype of antihousing; these chains are found in IgA, IgD, IgE, IgG, and IgM antibodies, respectively.

Distinct heavy chains differ in size and composition; α and γ contain approximately 450 amino acids and δ approximately 500 amino acids, while μ and 68 have approximately 550 amino acids. Each heavy chain has two regions, the constant region (CH) and the variable region (VH). In one species, the constant region is essentially identical in all antibodies of the same isotype, but differs in antibodies of different isotypes. Heavy chains γ, αand 67 have a constant region composed of three tandem Ig domains, and a hinge region for added flexibility; heavy chains μ and ε have a constant region composed of four immunoglobulin domains. The variable region of the heavy chain differs in antibodies produced by different B cells, but is the same for all antibodies produced by a single B cell or B cell clone. The variable region of each heavy chain is approximately 110 amino acids long and is composed of a single Ig domain.

In mammals, there are two types of immunoglobulin light chain denoted by λ and κ . A light chain has two successive domains: one constant domain (CL) and one variable domain (VL). The approximate length of a light chain is 211 to 217 amino acids. Each antihousing contains two light chains that are always identical; only one type of light chain, κ or λ, is present per antihousing in mammals.

Although the general structure of all antibodies is very similar, the unique property of a given antihousing is determined by the variable (V) regions, as detailed above. More specifically, variable loops, three each the light (VL) and three on the heavy (VH) chain, are responsible for binding to the antigen, i.e. for its antigen specificity. These loops are referred to as the Complementarity Determining Regions (CDRs). Because CDRs from both VH and VL domains contribute to the antigen-binding site, it is the combination of the heavy and the light chains, and not either alone, that determines the final antigen specificity.

An “antihousing fragment” contains at least one antigen binding fragment as defined above, and exhibits essentially the same function and specificity as the complete antihousing of which the fragment is derived from. Limited proteolytic digestion with papain cleaves the Ig prototype into three fragments. Two identical amino terminal fragments, each containing one entire L chain and about half an H chain, are the antigen binding fragments (Fab). The third fragment, similar in size but containing the carboxyl terminal half of both heavy chains with their interchain disulfide bond, is the crystalizable fragment (Fc). The Fc contains carbohydrates, complement-binding, and FcR-binding sites. Limited pepsin digestion yields a single F(ab′)2 fragment containing both Fab pieces and the hinge region, including the H-H interchain disulfide bond. F(ab′)2 is divalent for antigen binding. The disulfide bond of F(ab′)2 may be cleaved in order to obtain Fab′. Moreover, the variable regions of the heavy and light chains can be fused together to form a single chain variable fragment (scFv).

Pharmaceutically acceptable salts are for example acid addition salts and basic salts. Acid addition salts are e.g. HCI or HBr salts. Basic salts are e.g. salts having a cation selected from alkali or alkaline, e.g. Na+, or K+, or Ca2+, or an ammonium ion N+(R1)(R2)(R3)(R4), wherein R1 to R4 independently of each other mean: hydrogen, an optionally substituted C1-C6-alkyl group, an optionally substituted C2-C6-alkenyl group, an optionally substituted C6-C10-aryl group, or an optionally substituted C6-C10-heteroaryl group. Further examples of pharmaceutically acceptable salts are described in “Remington's Pharmaceutical Sciences” 17. ed. Alfonso R. Gennaro (Ed.), Mark Publishing Company, Easton, Pa., U.S.A., 1985 and in Encyclopedia of Pharmaceutical Technology.

Pharmaceutically acceptable solvates are for example hydrates.

An exemplary embodiment of the invention will now be described with reference to the accompanying drawings in which:

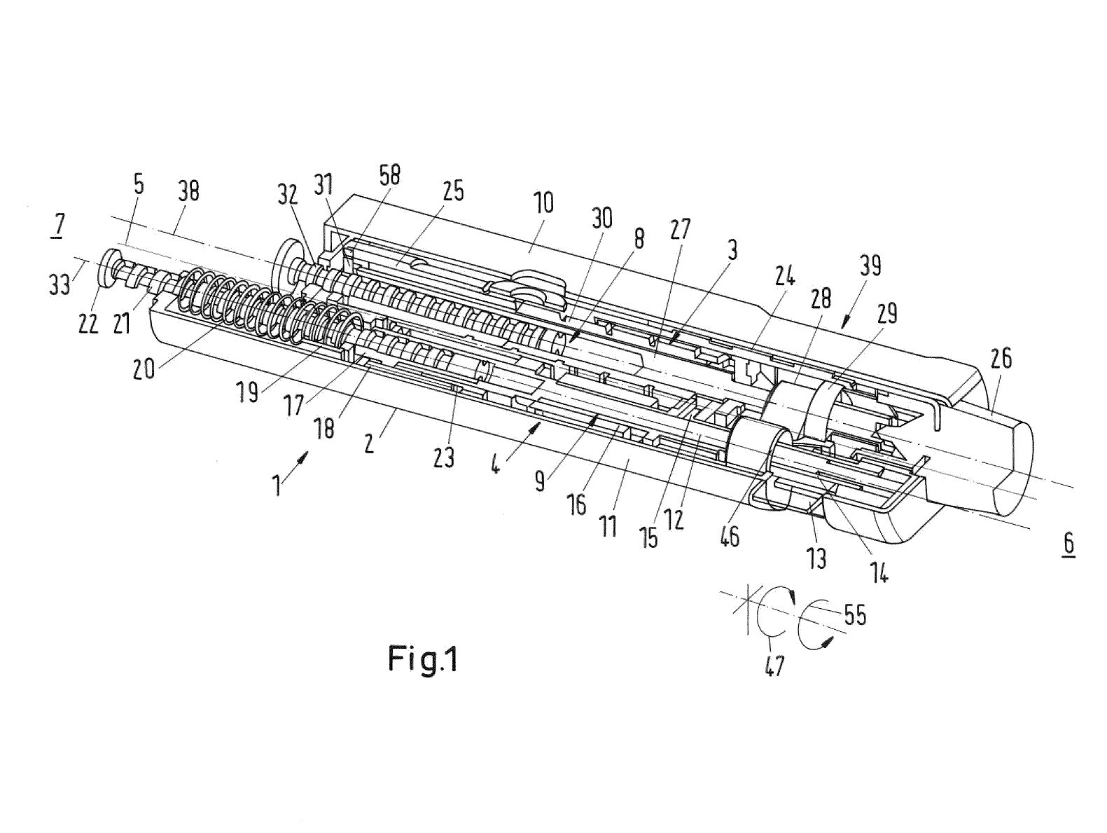

FIG. 1 shows in a perspective sectional view a drug delivery device in accordance with the present invention;

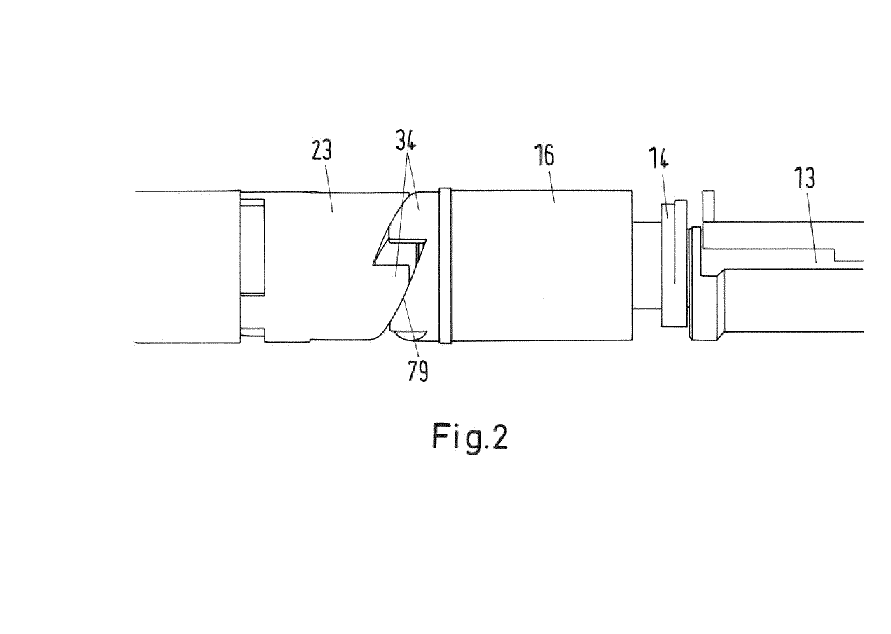

FIG. 2 shows element s of the drug delivery device in a side view;

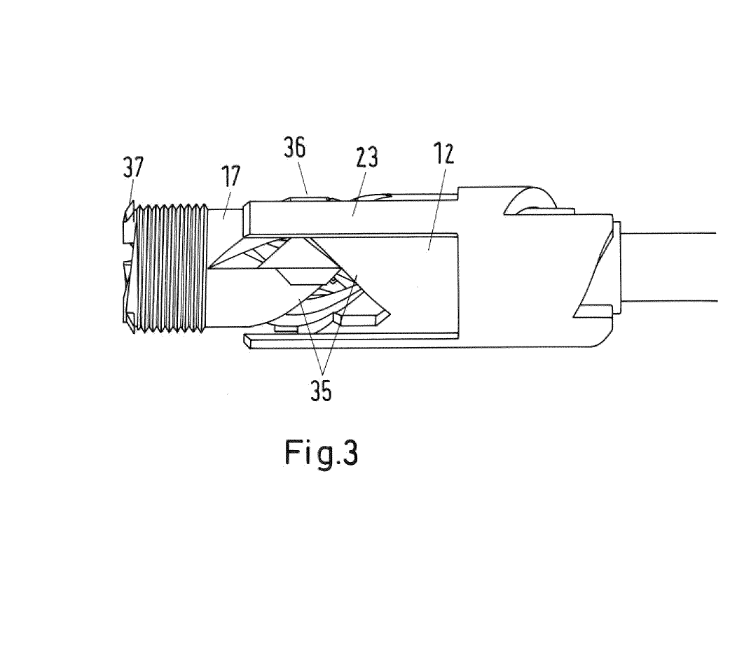

FIG. 3 shows further elements of the drug delivery device in a side view;

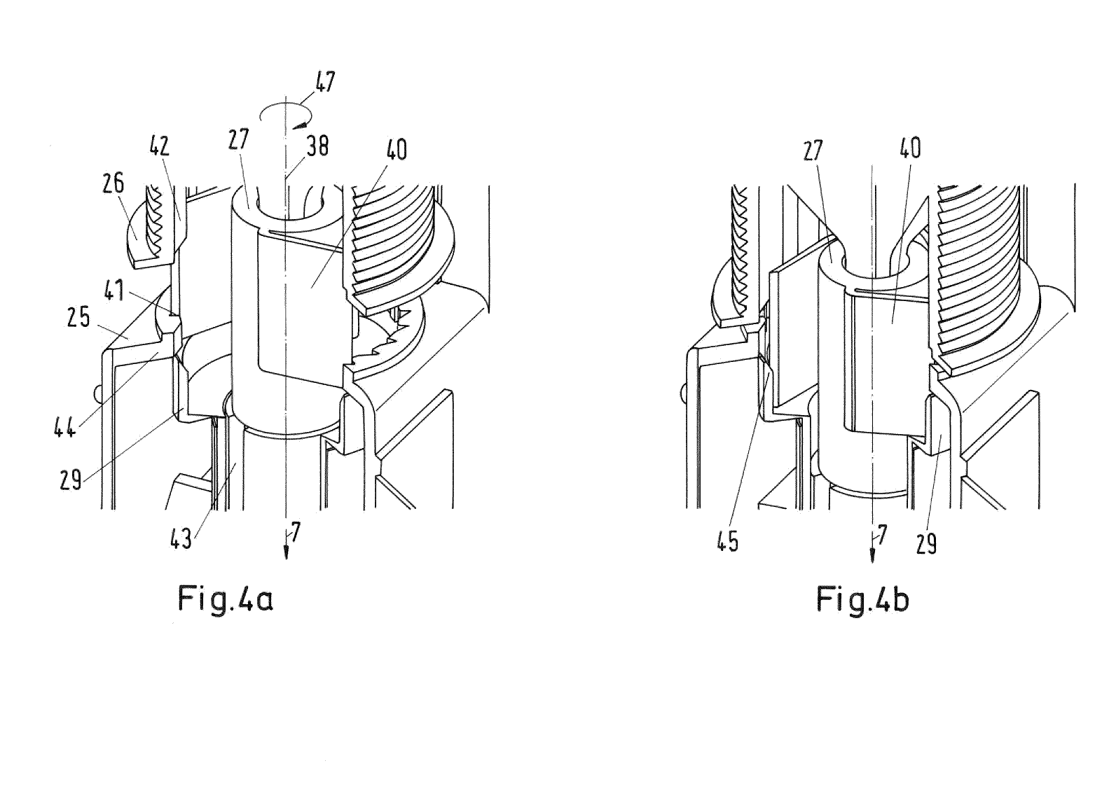

FIG. 4a, 4b show in a perspective sectional view elements of the primary drug delivery assembly;

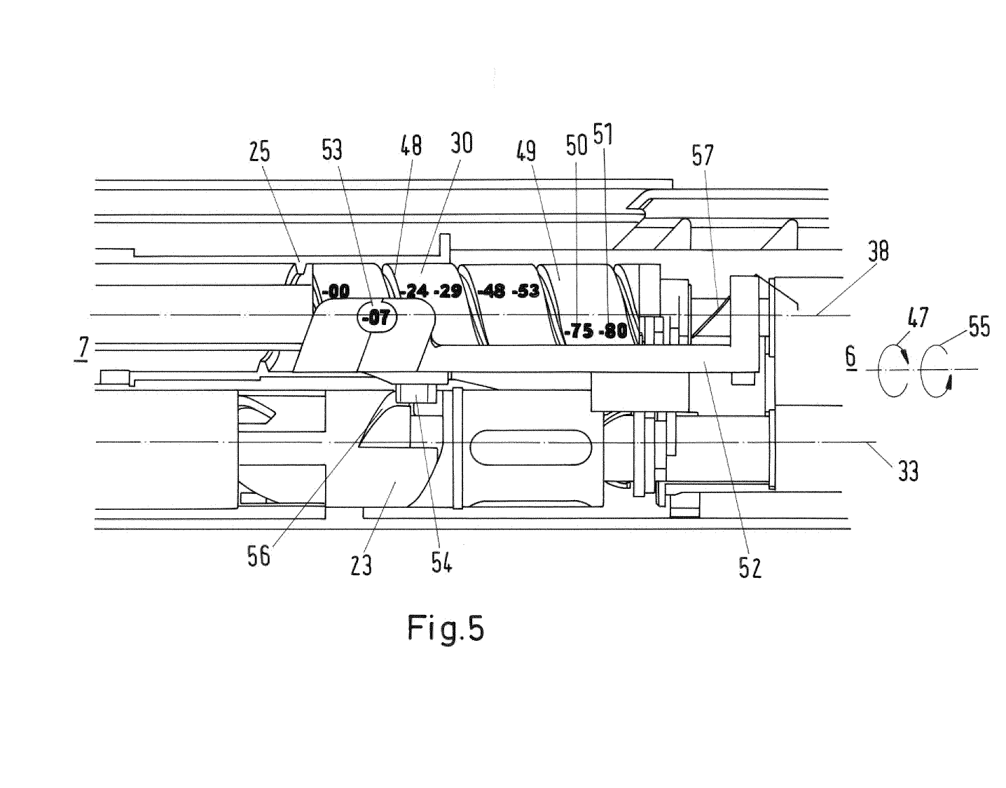

FIG. 5 shows the interior of a section of the drug delivery device;

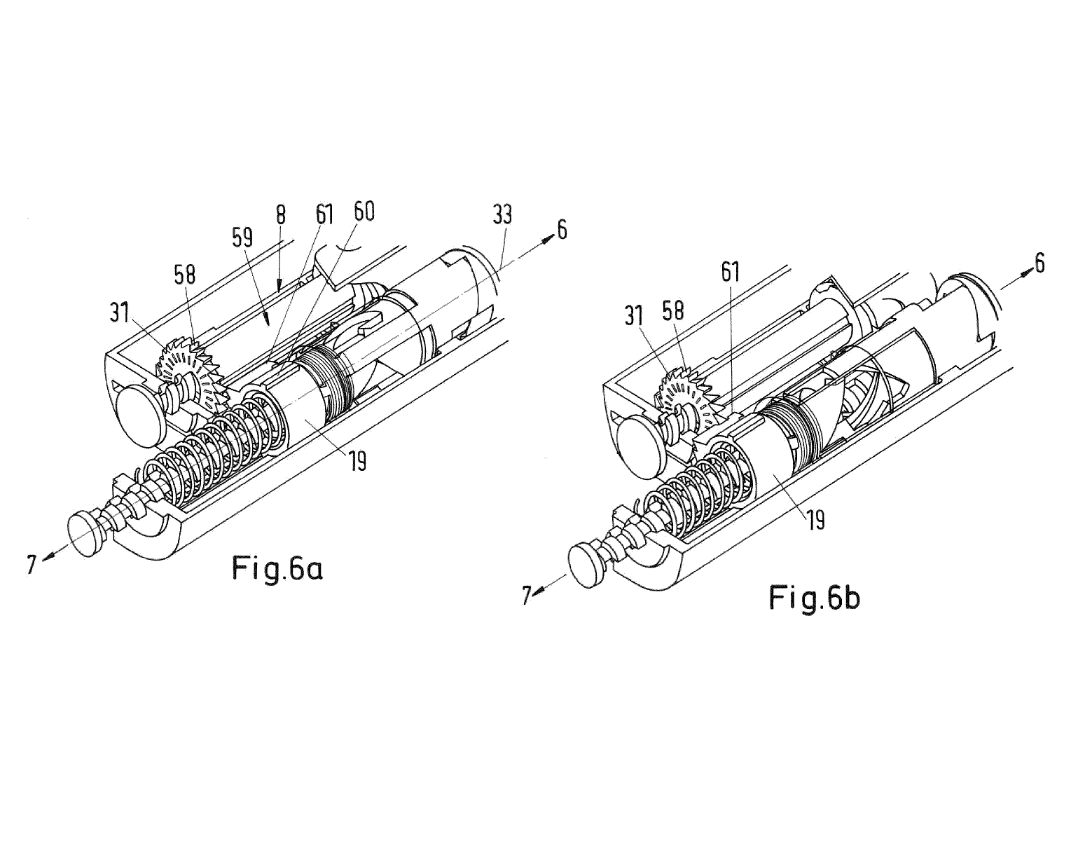

FIG. 6a, 6b show in a perspective view another section of the drug delivery device;

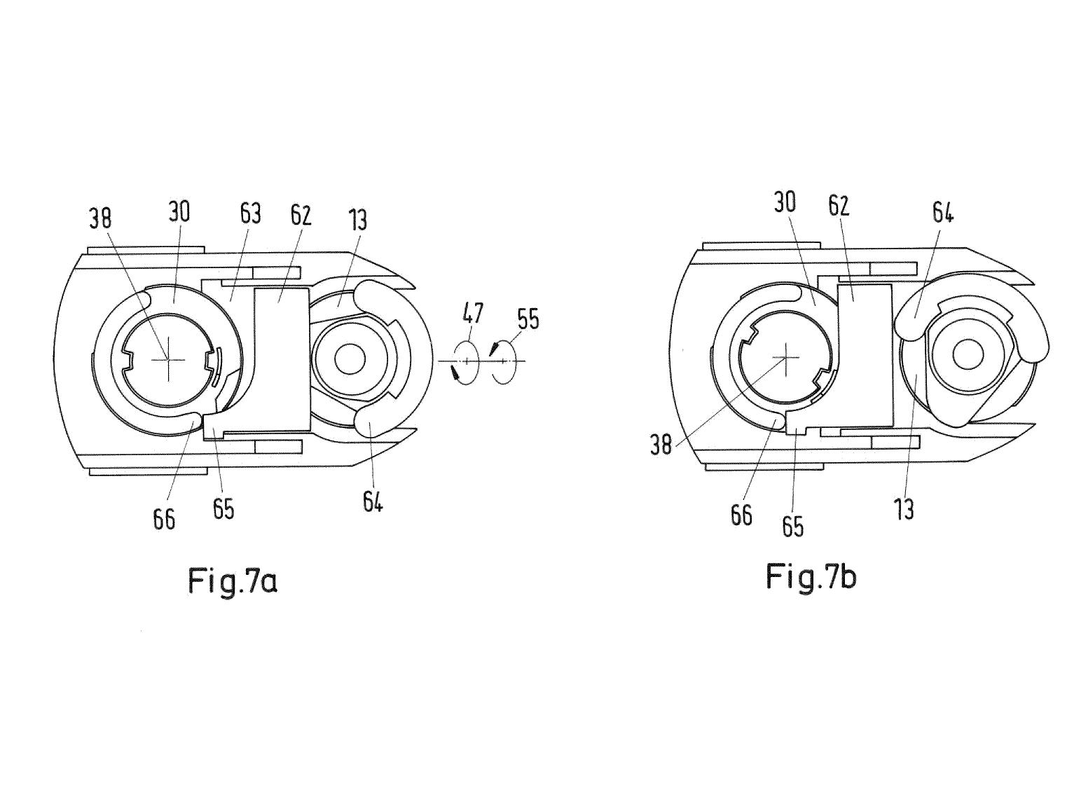

FIG. 7a, 7b show in a plane view another section of the drug delivery device;

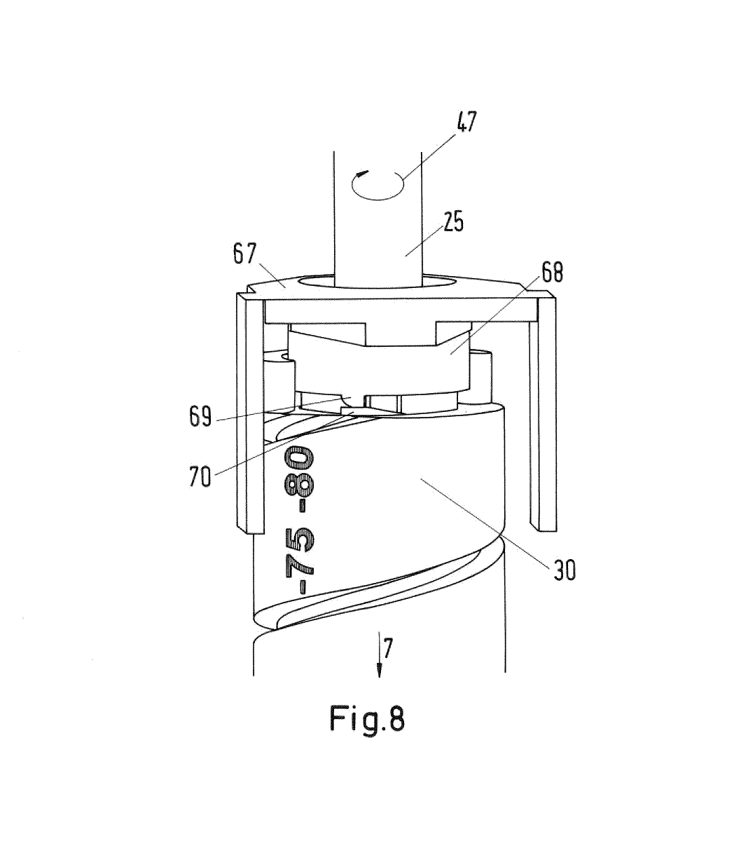

FIG. 8 shows in a perspective view another part of the drug delivery device;

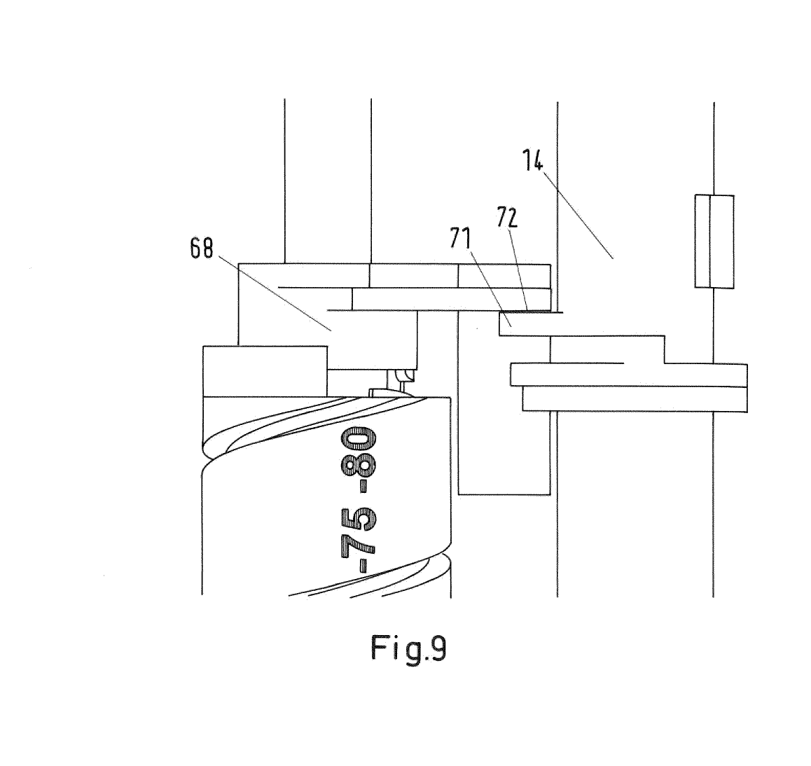

FIG. 9 shows in a side view another part of the drug delivery device;

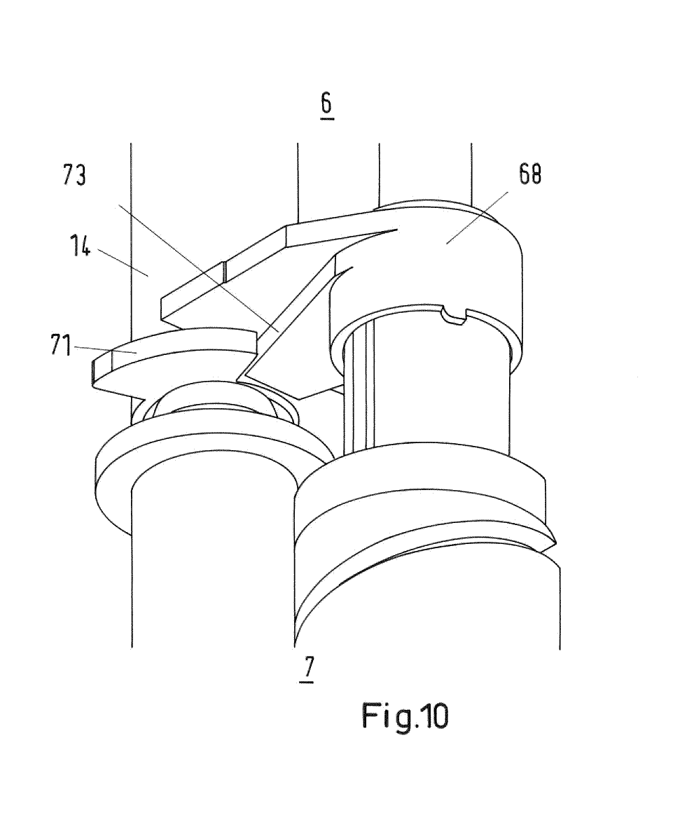

FIG. 10 shows in a perspective view another part of the drug delivery device;

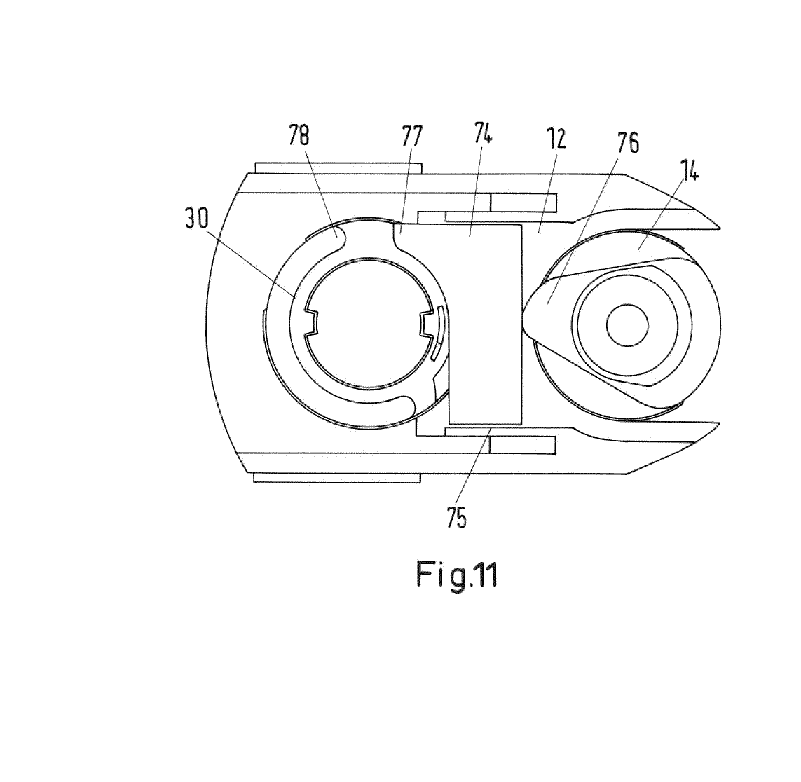

FIG. 11 show in a plane view another section of the drug delivery device;

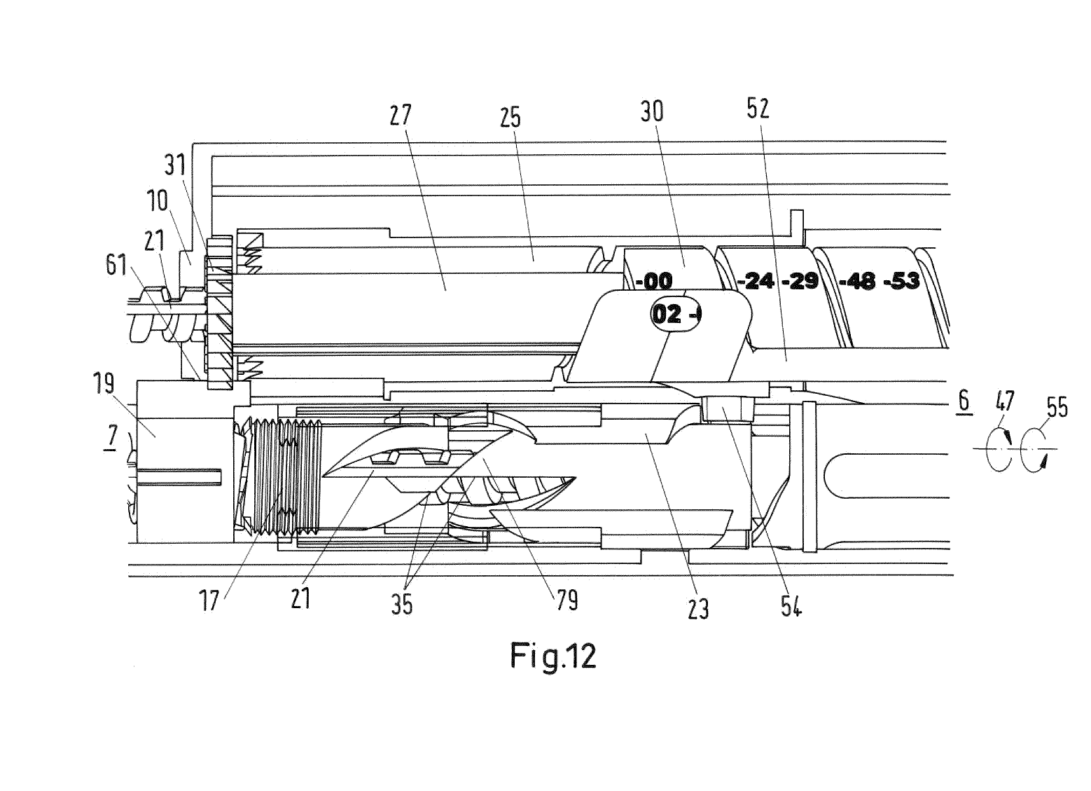

FIG. 12 shows the interior of another section of the drug delivery device in a side view

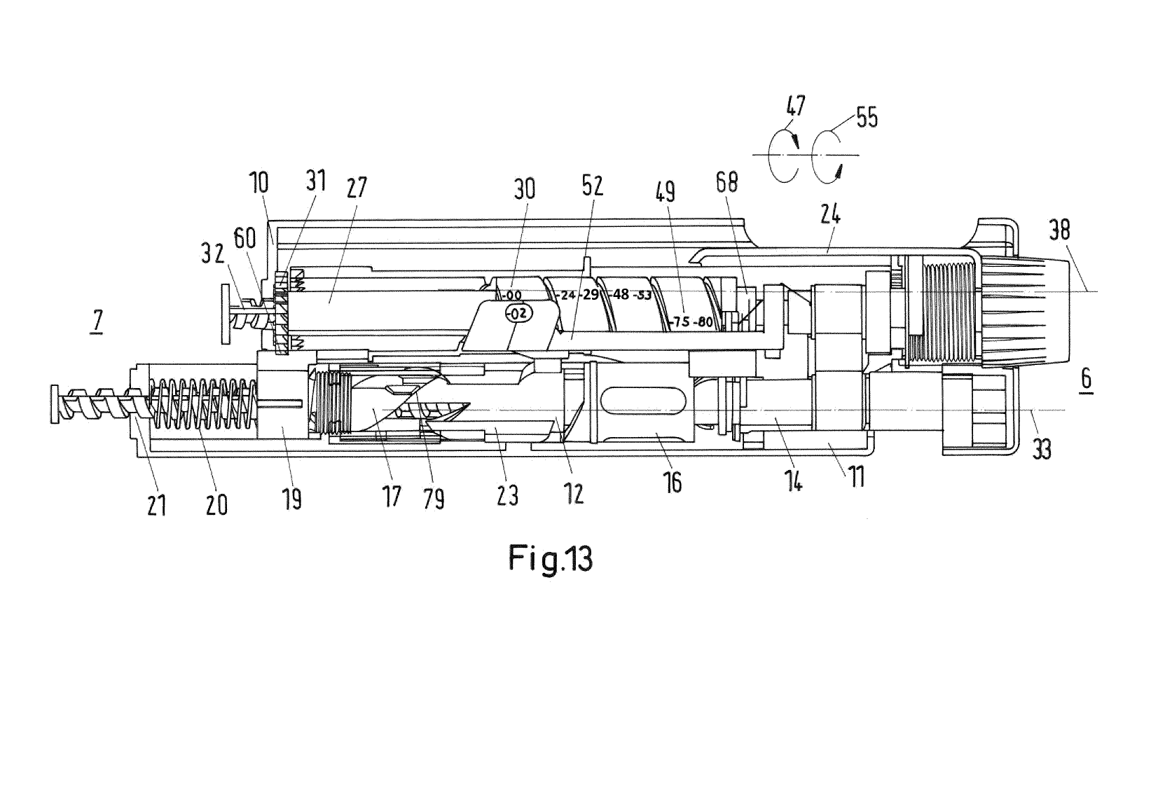

FIG. 13 shows the interior of the drug delivery device.

The drug delivery device 1 comprises a drug delivery device housing 2 in which a primary drug delivery assembly 3 and a secondary drug delivery assembly 4 are retained. The drug delivery device housing 2 extends along a longitudinal axis 5 from a proximal end 6 to a distal end 7. Each of the drug delivery assemblies 3, 4 includes an individual dose setting mechanism and dose dispensing mechanism. The primary drug delivery assembly 3 comprises a variable dose setting and dispensing mechanism 8, while the secondary drug delivery assembly 4 comprises a fixed dose setting and dispensing mechanism 9. The drug delivery device housing 2 includes two housing elements, a primary drug delivery assembly housing 10 and a secondary drug delivery assembly housing 11.

The secondary drug delivery assembly 4 includes the secondary drug delivery assembly housing 11, a secondary inner body 12, a selection element formed as a selection lever 13, a selection shaft 14, a selection spring 15, a selection sleeve 16, a secondary drive sleeve 17, a last dose nut 18, a ratchet 19, herein referred as the drive ratchet, a secondary dispense spring 20, a secondary lead screw 21, a bearing 22 and a setting sleeve 23.

The primary drug delivery assembly 3 includes the primary drug delivery assembly housing 10, a trigger 24, a primary inner body 25, a dial grip 26, a primary last dose nut (not shown), a primary drive sleeve 27, a primary dispense spring 28, a spring spool 29, a primary dose dial sleeve, herein referred to as the number sleeve 30, a clutch 31, a primary lead screw 32. At the distal end of the primary lead screw, a bearing is provided.

In the secondary drug delivery assembly 4, the secondary inner body 12 is rigidly constrained to the secondary drug delivery assembly housing 11 and provides together with the housing 11 a common principal axis 33.

The selection shaft 14 is axially constrained to the secondary inner body 12 and is free to rotate about the principal axis 33. The selection spring 15 acts between the secondary drug delivery assembly housing 11 and the selection shaft 14 to provide a restoring torque which returns the selection shaft to an “At Rest” rotational position. The selection lever 13 is rotatable from an intermediate “At Rest” position, respectively first position, in a first direction in counterclockwise direction 55 when viewed from the proximal end 6 to the distal end 7 into a second position. Further, the selection lever 13 is rotatable from the intermediate “At Rest” position, respectively first position, in a second direction in clockwise direction 47 when viewed from the proximal end 6 to the distal end 7 into a third position.

With respect to FIG. 2, the selection lever 13 is axially and rotationally constrained to the selection shaft 14. Its rotation is constrained by abutments with the secondary drug delivery assembly housing 11, which limits the rotation of the selection lever 13, and therefore the selection shaft 14.

The selection sleeve 16 is rotationally constrained to the selection shaft 14. It is free to move axially between abutments which limit its range of axial travel relative to the selection shaft 14. It has integrated sprung elements (not shown), which act to force the selection sleeve 16 towards its extreme distal location. The selection sleeve 16 may feature a set of markings which are visible to the user through an aperture in the drug delivery device housing. The marking displayed is dependent on the rotational position of the selection sleeve 16. The setting sleeve 23 is free to move axially along the secondary inner body. Its rotation is constrained by abutments with the secondary inner body. It interfaces with the selection sleeve 16 via a set of axial ratchet teeth 34, such that when the ratchet teeth 34 are fully engaged, the setting sleeve 23 is forced to rotate when the selection sleeve 16 rotates in a counterclockwise direction.

As shown in FIG. 3, the secondary drive sleeve 17 interfaces with the secondary inner body 12 via a set of mating axial ratchet teeth 35 such that the secondary drive sleeve 17 is prevented from rotating clockwise when the axial ratchet teeth 35 are fully engaged. The secondary drive sleeve 17 also interfaces with the setting sleeve 23 via a set of opposing axial ratchet teeth 36, such that counterclockwise rotation of the setting sleeve 23 forces the secondary drive sleeve 17 to move axially towards the distal end of the device.

The selection shaft 14, the selection sleeve 16 and the setting sleeve 23 form a coupling mechanism to transfer rotational movement of the setting element from the first into the second position into axial motion of the secondary drive sleeve 17 in distal direction.

The secondary last dose nut 18 (see FIG. 1) is rotationally constrained to the setting sleeve 23 via a splined interface, which allows the secondary last dose nut 18 to move axially. The secondary last dose nut 18 is threaded to the secondary drive sleeve 17, such that clockwise rotation of the secondary drive sleeve 17 relative to the setting sleeve 23 causes the secondary last dose nut to advance towards an abutment 37 (FIG. 3) at the distal end of the secondary drive sleeve 17.

The drive ratchet 19 is rotationally constrained to the secondary drug delivery assembly housing 11 via a splined interface, which allows the drive ratchet 19 to move axially. The drive ratchet 19 interfaces with the secondary drive sleeve 17 via a set of mating axial ratchet teeth that prevent counterclockwise rotation of the secondary drive sleeve 17 when the ratchet teeth are engaged.

The secondary dispense spring 20 constitutes a biasing element that is located between a distal surface of the secondary drug delivery assembly housing 11 and the drive ratchet 19 and biases the ratchet 19 in proximal direction.

The secondary lead screw 21 is rotationally constrained to the secondary drive sleeve 17 via a splined interface and is threaded to the secondary drug delivery assembly housing 11 such that clockwise rotation of the secondary lead screw 21 causes the secondary lead screw 21 to advance towards the distal end 7.

The bearing 22 is axially constrained to the secondary lead screw 21 to create an abutment with a bung within a medicament cartridge that contains a secondary medicament, wherein the medicament cartridge is attachable to the distal end the of the secondary drug delivery assembly housing 11. As the secondary lead screw 21 advances in distal direction, the bearing 22 displaces the bung, which causes liquid medicament to be displaced from the cartridge and to be dispensed through an injection needle attached to the distal end of the cartridge.

In the primary drug delivery assembly 3 (FIG. 1), the primary inner body 25 is rigidly constrained to the primary drug delivery assembly housing 10 and provides together with the primary drug delivery assembly housing 10 a common principal longitudinal axis 38 for the components of the variable dose setting and dose dispensing mechanism 8 which are located concentrically with respect to said axis 38.

The trigger 24 is located between the primary drug delivery assembly housing 10 and the primary inner body 25 and is accessible to user via an aperture 39 in the primary drug delivery assembly housing 10. The trigger 24 can be depressed by the user, moving it towards the principal axis 38. The trigger 24 engages with angled splines in the primary inner body 25 such that as the trigger 24 is depressed, it moves towards the distal end 7 of the device 1.

The dial grip 26 is located at the proximal end 6 of the drug delivery device 1 and protrudes through a proximal aperture in the drug delivery device housing 2 so that the dial grip 26 is accessible to the user. The dial grip 26 is axially constrained to the trigger 24 such that as the trigger 24 is depressed, the dial grip 26 moves toward to the distal end 7. As the dial grip 26 moves axially in proximal direction, it engages with splines (not shown) on the primary drug delivery assembly housing 10 which constrain its rotation. When the trigger 24 is ‘At Rest’, meaning that is is not forced proximally by engagement with the primary inner body 25, the dial grip 26 can be rotated around the longitudinal axis 38 by the user.

The primary last dose nut is rotationally constrained by the trigger 24. It engages with the dial grip 26 via a threaded interface such that the primary last dose nut advances towards an abutment at the proximal end 6 of the device as the dial grip 26 is rotated clockwise.

With respect to FIG. 4a, the primary drive sleeve 27 features a series of flexible ratchet arms 40 that interface with ratchet teeth 41 in the primary inner body 25 and ratchet teeth 42 in the dial grip 26. The ratchet interface between the primary drive sleeve 27 and the dial grip 26 causes the primary drive sleeve 27 is rotated with the dial grip 26 when the dial grip 26 is rotated around the axis 38 in clockwise direction as indicated by the arrow 47. When the primary drive sleeve 27 rotates clockwise the flexible ratchet arms 40 advance over the ratchet teeth 41 in the primary inner body 25.

If the dial grip 26 is rotated counterclockwise, the flexible ratchet arms 40 of the primary drive sleeve 27 are deflected by the ratchet teeth 42 on the dial grip 26. The primary drive sleeve 27 is initially prevented from rotating by the engagement of the flexible arms 40 with the ratchet teeth 41 on the primary inner body 25. Before the flexible ratchet arms 40 advance over the teeth in the dial grip 26, the arms 40 deflect sufficiently such as to remove the engagement with the teeth 41 on the primary inner body 25. The ratchet between the primary drive sleeve 27 and primary inner body can now be overhauled and the primary drive sleeve 27 rotates counterclockwise with the dial grip 26. The spacing of the ratchet teeth in the dial grip 26 and the primary inner body 25 provides a detented position for the dial grip 26 and the primary drive sleeve 27 corresponding to each dose unit.

The axial position of the primary drive sleeve 27 is constrained by an abutment with the dial grip 26 such that it moves towards the distal end 7 of the device 1 when the trigger 24 is depressed. The spring spool 29 interfaces with the primary drive sleeve 27 via a splined interface 43 which prevents relative rotation. The spring spool 29 is axially constrained via an abutment 44 to the primary inner body 25.

As shown in FIG. 4b, when the primary drive sleeve 27 is displaced axially in distal direction by depressing the trigger, the flexible ratchet arms 40 on the primary drive sleeve 27 engage with an inclined collar 45 on the spring spool 29 which causes the flexible arms 40 to deflect radially inwardly. When the ratchet arms 40 are deflected, the ratchet engagements with the primary inner body 25 and the dial grip 26 are removed and the primary drive sleeve 27 is able to rotate independently of these components.

The primary dispensing spring 28 (FIG. 1) is a constant torque motor spring. The primary dispensing spring 28 is formed to a nominal radius and is wound onto a storage spool 46 whose radius is larger than the nominal radius of the spring 28. The primary dispensing spring 28 therefore naturally winds itself tightly to the storage drum. The storage spool 46 fits concentrically around the selection shaft 14. The primary dispensing spring 28 is extended from the storage spool 46 and attaches via an anchorage to the spring spool 29. As the spring spool 29 is rotated clockwise (indicated by the arrow 47), the primary dispensing spring 28 is wound around the spring spool 29 and is forced to unwind from the storage spool 46. Unwinding the primary dispensing spring 28 from the storage spool 46 applies a torque to the spring spool 29, which via its spline engagement 43 with the primary drive sleeve 27 applies a torque to the primary drive sleeve 27. When a dose in the primary drug delivery assembly has not been set, a nominal number of turns of the primary dispensing spring 28 are wound onto the spring spool 29 such that the torque applied to spring spool 29 remains constant as the spring spool 29 is rotated in clockwise direction 47.

The number sleeve 30 interfaces with the primary drive sleeve 27 via a splined interface which constrains the rotation of the number sleeve 30 to that of the primary drive sleeve 27. As shown in FIG. 5, the number sleeve 30 interfaces with the primary inner body 25 via a threaded interface 48, which causes the number sleeve 30 to move along a helical path as the primary drive sleeve 27 is rotated. The orientation of the threaded interface 48 causes the number sleeve 30 to advance towards the distal end 7 as the primary drive sleeve 27 is rotated in clockwise direction 47. The number sleeve 30 is marked with indices 49 in the form of numbers corresponding to the dose position of the device. There are two sets of number markings. The first number marking 50 corresponds to the variable dose dialed when the fixed dose is not selected and the second number marking 51 corresponds to the increased overall dose when the fixed dose is selected.

A moveable element, herein referred to as the lens 52 is constrained to move axially relative to the primary inner body 25, wherein its axial position in proximal and distal direction is limited by abutments. An abutment with the primary drive sleeve 27 is generated when the trigger 24 is depressed, which limits the proximal position of the lens 52 during dispensing.

The lens 52 includes a transparent aperture 53, which aligns to an elongated open aperture in the primary inner body 25 such that the indices on the number sleeve 30 are visible to the user through the transparent aperture 53. The axial position of the lens 52 determines which set of number markings, the first number marking 50 or the second number marking 51, is visible to the user. The lens 52 interfaces via an abutment 54 with the setting sleeve 23. As the setting sleeve 23 is rotated in counterclockwise direction 55 to set a dose in the secondary drug delivery assembly around the axis 33, a counterabutment surface 56 abuts the abutment 54 and causes the lens 52 to move in proximal direction. When the lens 52 moves in distal direction as a result of a distal movement of the trigger 24 during dose dispense of the fixed dose, abutment with the setting sleeve 23 causes the setting sleeve 23 to advance towards the distal end 7.

A lens spring 57 is fitted around the lens 52 and provides an axial force between the primary inner body 25 and the lens 52, which acts to force the lens 52 in distal direction.

The clutch 31 (FIG. 1) is located at the distal end of the primary inner body 25 and engages the primary inner body 25 via a set of axial ratchet teeth 58. An axial spring element is integrated into the primary drug delivery assembly housing 10 to bias the clutch 31 in proximal direction to engage with the primary inner body 25. When the clutch 31 is engaged with the primary inner body 25, both components are rotationally constrained to each other.

The clutch 31 interfaces with the primary drive sleeve 27 when the primary drive sleeve 27 advances towards the distal end 7 of the device 1 via a set of axial ratchet such that as the primary drive sleeve 27 moves distally, the engagement of the axial ratchet teeth causes the clutch 31 to move axially and the interface with the primary inner body 25 is removed. At this point, the clutch 31 is axially and rotationally constrained to the primary drive sleeve 27. The clutch 31 features a set of radial ribs on the surface on which the axial spring element of the primary drug delivery assembly housing 10 acts. These ribs generate a click as the clutch rotates corresponding to each dose unit dispensed.

The primary lead screw 32 is rotationally constrained to the clutch 31 via a splined interface. It is threaded to the primary drug delivery assembly housing 10 such that counterclockwise rotation 55 of the primary lead screw 32 causes the primary lead screw 32 to advance in distal direction such that a bearing at the distal end of the primary lead screw 32 may create an abutment with a bung within a cartridge which may be attached to the distal end of the primary drug delivery assembly housing 10 and which may contain a primary medicament. As the primary lead screw 32 advances in distal direction, the bearing displaces the bung, which causes liquid primary medicament to be dispensed through an attachable injection needle.

As shown in FIGS. 6a and 6b, the drug delivery device 1 includes a first interlock or lock mechanism 59 that is configured to lock the dispensing function of the variable dose setting and dose dispensing mechanism 8. The lock mechanism 59 is activated during dose setting in the secondary drug delivery assembly and provides an efficient interlock between the variable dose setting and dose dispensing mechanism and the fixed dose setting and dose dispensing mechanism so that the actions a user may take are restricted thereby increasing the safety of the device.

The lock mechanism 59 includes the drive ratchet 19 and the clutch 31. The drive ratchet 19 is in a splined interface 60 with the secondary drug delivery assembly housing such that the drive ratchet 19 is axially moveable but cannot rotate around the axis 33. The axial position of the drive ratchet 19 is dependant upon the state of the fixed setting mechanism of the secondary drug delivery assembly. When the drive ratchet 19 is in its extreme proximal position, which corresponds to the fixed dose mechanism not being set to deliver a dose as shown in FIG. 6a, the drive ratchet 19 provides no locking function.

When secondary drug delivery assembly is actuated to set a fixed dose (FIG. 6b), the drive ratchet 19 moves towards the distal end of the device such that a recessed surface 61 with axially extending grooves is engaged by the ratchet teeth 58 on the outer surface of the clutch 31. This causes a splined interface between the drive ratchet 19 and the clutch 31. Thereby, the clutch 31 is locked against rotation.

This splined interface is maintained until the fixed dose setting and dispensing mechanism is reset or the fixed dose is dispensed, at which point the drive ratchet 19 returns to its proximal position as shown in FIG. 6a. Whilst this spline interface is maintained, the variable dose setting and dispensing mechanism is prevented from dispensing, as the clutch 31 is unable to rotate. Via this lock mechanism, the fixed dose will be dispensed prior to the delivery of the variable dose.

As shown in FIG. 7a, which corresponds to a view from the distal end of the device in proximal direction, a sliding element 62 is arranged between the selection element 13, which is in the intermediate “At Rest” position, and the number sleeve 30. The sliding element 62 is a transfer element and is configured to engage the number sleeve 30 such that lateral motion of the transfer element causes rotation of the number sleeve 30. The sliding element 62 is constrained to slide within a slot 63 in the drug delivery device housing and is activated by interaction with the selection lever 13 which comprises a profiled abutment surface 64 that has an increased radius compared to a radially opposed section of the selection lever 13.

The sliding element 62 is basically L-shaped and has an engagement section 65 projecting towards the number sleeve 30 which has a counter engagement surface 66. As the selection lever 13 is rotated in clockwise direction 47 or in counterclockwise direction 55, from its ‘At Rest’ position, the abutment between abutment surface 64 and the sliding element 62 forces the sliding element 62 to move laterally towards the axis 38. Abutment between the sections 65 and 66 forces the number sleeve 30 to rotate to a two unit position as shown in FIG. 7b in clockwise direction. The sliding element 62 may be therefore referred to as the two units advance slider.

A second interlock mechanism 67 is shown in FIG. 8 and functions as a latching mechanism. This second interlock mechanism functions when the selection element 13 is in the second or third position, which corresponds to a rotation of the setting element 13 from the intermediate position in clockwise or counterclockwise direction. The second interlock mechanism 67 comprises a latch 68 that is rotationally constrained by the primary inner body 25. The axial position of the latch 68 is determined by the action of the lens spring which may be arranged between the latch 68 and the lens and abutments which occur with the selection shaft and the number sleeve 30.

The lens spring acts to force the latch 68 in distal direction 7. With the device resp. the selection element in the ‘At Rest’ state, an abutment occurs between the latch 68 and the number sleeve 30, limiting the latch's travel in the distal direction. The latch 68 comprises a distal projection 69 and the number sleeve 30 has a proximal projection 70 with a slanted surface. When number sleeve 30 is rotated this abutment condition is removed.

As shown in FIG. 9, the selection shaft 14 comprises a profiled boss 71 which provides a second axial abutment 72 between the latch 68 and the selection shaft 14 when the selection shaft 14 is in the ‘At Rest’ position which corresponds to the intermediate position of the selection element. When the selection shaft 14 is rotated to either set or not set the fixed dose, the axial abutment with the profiled boss 71 is removed.

When the selection shaft 14 is rotated and the number sleeve is advanced to the two unit position as explained above, the latch 68 is able to travel to its extreme distal position as shown in FIG. 10. In this position, the latch 68 provides a radial abutment 73 with the profiled boss 71 of the selection shaft 14, such that the selection shaft 14 is prevented from rotating back to its ‘At Rest’ location.

This interlock maintains the selection shaft 14 in either of its rotated positions until the number sleeve 30 returns to the zero dose position as shown in FIG. 8. As the number sleeve 30 with the proximal projection 70 re-engages with the distal projection 69, the latch 68 is moved proximally and the radial abutment between the selection shaft 14 and the latch 68 is removed.

In FIG. 11, a further interlock mechanism is shown which includes a two units interlock slider 74 and functions as a limiting mechanism. The interlock slider 74 is constrained to slide within a slot 75 in the secondary inner body 12. It is packaged between the number sleeve 30 and the selection shaft 14. The selection sleeve 14 has a cam profile 76 on its outer radial surface. When the selection shaft is in the “At Rest” position as shown in FIG. 11, the cam profile 76 constrains the interlock slider 74 in a lateral position in which a projection 77 is located such that rotation of the number sleeve 30 in clockwise direction causes engagement of the projection 77 and an abutment surface 78 on the number sleeve 30 such that the number sleeve 30 cannot be rotated further in clockwise direction. The abutment with the number sleeve 30 limits the rotation of the number sleeve to two units (equating to a prime dose).

Rotation of the selection shaft 14 is required to allow the number sleeve 30 to advance against its abutment with the interlock slider 74. This interlock therefore limits the selectable dose on the variable dose setting and dispensing mechanism to two units unless the user chooses to either set or not set the fixed dose by rotation the selection shaft 14 with the selection element. When the selection shaft 14 has been rotated, the number sleeve 30 may be advanced beyond the two unit dose position, as the number sleeve 30 is able to displace the interlock slider 74 towards the selection shaft 14.

In the following, it is described how a fixed dose in the fixed dose setting and dispensing mechanism can be selected with reference to FIG. 1.

If the user chooses to set the fixed dose to be dispensed, the selection lever 13 is rotated in counterclockwise direction 55, when viewed from the proximal 6 end of the device 1 to the distal end 7 of the device 1, up to an abutment with the secondary drug delivery assembly housing 11 from the first (intermediate) position around the longitudinal axis 33 of the secondary drug delivery assembly 4 into the second position.

The selection shaft 14, the selection sleeve 16, the setting sleeve 23 and the secondary last dose nut 18 rotate with the selection lever 13. The secondary drive sleeve 17 does not rotate and moves axially towards the distal end 7 of the device 1. Relative rotation between the secondary drive sleeve 17 and the secondary last dose nut 18 causes the secondary last dose nut 18 to advance towards its abutment, which engages when the fixed dose mechanism has dispensed a defined number of doses. When the abutment engages, the user is unable to set the fixed dose to be dispensed.

Rotation of the selection sleeve 16 causes rotation of the setting sleeve 23 which advances the secondary drive sleeve 17 axially. Axial movement of the secondary drive sleeve 17 displaces the drive ratchet 19, which compresses the secondary dispense spring 20.

Counterclockwise rotation of the selection sleeve 16 causes the marking on the selection sleeve 16 displayed to the user through the aperture in the secondary drug delivery housing 11 to change. The marking may change colour to identify to the user that the fixed dose mechanism is set to dispense.

Counterclockwise rotation of the setting sleeve 23 causes the lens 52 to move towards its proximal position, so that the alternative second set of number markings 51 on the number sleeve is displayed to the user. Rotation of the selection lever 13 causes the two units advance slider to rotate the number sleeve 30 to the two unit position. Rotation of the selection shaft 14 allows the latch 68 to move axially into its distal position, creating an interlock which retains the selection shaft in its rotated position. At this point, the fixed dose has been selected and is set to be dispensed when the user depresses the trigger 24.

In the following, it is described how the user can choose not to set a fixed dose. In this case, the selection lever 13 is rotated clockwise from its intermediate “At Rest” position, namely in a direction opposite the direction in which the user rotates the selection element to set a fixed dose in the secondary drug delivery assembly. Here, the user rotates the selection element in a second direction from the first intermediate position in clockwise direction into the third position.

The selection shaft 14 and selection sleeve 16 rotate with the selection lever 13. The setting sleeve 23 remains stationary and the selection sleeve 16 moves axially towards the proximal end 6 of the device 1 as it slides up slopes faces or slanted surfaces 79 (FIG. 2) of the axial ratchet interface 34 with the setting sleeve 23.

Clockwise rotation of the selection sleeve 16 causes the marking displayed to the user through the aperture in the secondary drug delivery assembly housing to change. The marking may change color to identify to the user than the fixed dose mechanism is set not to dispense. Rotation of the selection lever 13 causes the two units advance slider to rotate the number sleeve 30 to the two unit position.

Rotation of the selection shaft 14 allows the latch to move axially into its distal position, thus creating an interlock which retains the selection shaft 14 in its rotated position. At this point, the user has selected not to dispense a fixed dose when the trigger 24 is depressed and may continue to set a variable dose.

To set a variable dose, the user rotates the dial grip 26 in clockwise direction 47. During setting of a dose, the primary drive sleeve 27, the number sleeve 30 and the spring spool 29 rotate with the dial grip 26. The flexible ratchet arms on the primary drive sleeve generate a click as the ratchet engagement with the primary inner body 25 is overhauled, corresponding to each dose unit.

Clockwise rotation of the dial grip 26 advances the primary last dose nut towards its abutment. When the primary last dose nut engages with its abutment, further rotation of the dial grip 26 is prevented. This occurs when the combination of the doses dispensed and the doses dialed reach a defined number, corresponding to the total number of doses available from the cartridge. Clockwise rotation of the spring spool 29 acts to wind up the primary dispensing spring 28, which stores energy to dispense the dose.

Rotation of the number sleeve 30 is limited to two units by the two units interlock slider as explained with regard to FIG. 11. If the user has moved the selection lever 13 to either of its set positions, the number sleeve is able to move the two units interlock slider laterally such that number sleeve 30 is allowed to be rotated beyond the two unit position. The user is then able to continue to rotate the dial grip 26 until the required variable dose is set. The set dose is displayed to the user through the aperture 39 in the primary drug delivery assembly housing 11 and the lens 52 which align to make the appropriate set of number markings 50 or 51 (FIG. 5) visible. At this point, a variable dose has been set to be dispensed when the user depresses the trigger 24.

To unset a dose, the user rotates the dial grip 26 in counterclockwise direction 55. During unsetting of a dose, the primary drive sleeve 27, the number sleeve 30 and the spring spool 29 rotate with the dial grip 26. Counter clockwise rotation of the dial grip 26 causes the flexible ratchet arms on the primary drive sleeve 27 to deflect such that the ratchet engagement with the primary inner body 25 can be overhauled. The user can adjust the set variable dose between two units and the maximum dose without unsetting the fixed dose mechanism.

If the user chooses to fully reset the device, the dial grip 26 is rotated counter clockwise until the number sleeve 30 returns to the zero unit position. As the number sleeve 30 returns to the zero unit position, the axial abutment between the number sleeve 30 and the latch raises the latch towards its proximal position. As the latch rises, the selection shaft 14 is free to rotation and under the action of the selection spring 15 returns to its ‘At Rest’ position. Rotation of the selection shaft 14 allows the remaining components within the fixed dose setting and dispensing mechanism 9 to return to their ‘At Rest’ positions. At this point, the device has been returned to it's ‘At Rest’ state.

In the following, it is explained how a set fixed dose is dispensed with reference to FIG. 12. To dispense a dose, the user depresses the trigger such that the trigger moves laterally axially towards the distal end 7 of the device. As the trigger is depressed, the dial grip and the primary drive sleeve 27 move axially with the trigger. The dial grip becomes rotationally constrained to the primary drug delivery assembly housing 10 and the flexible ratchet arms on the primary drive sleeve 27 deflect to allow the primary drive sleeve 27 to rotate relative to the dial grip and primary inner body 25.

The lens 52 is displaced from its proximal position towards the distal end 7 by its abutment with primary drive sleeve 27. The abutment between the abutment 54 of the lens 52 and the setting sleeve 23 causes the setting sleeve 23, the secondary drive sleeve 17 and the drive ratchet 19 to be displaced in distal direction. Displacement of the drive ratchet 19 creates an engagement between the recessed surface 61 of the ratchet 19 and the ratchet teeth of the clutch 31 creating a splined interface and locking the primary dose setting and dose dispensing mechanism from dispensing. As the drive ratchet 19 is displaced, the secondary dispense spring is further compressed. The secondary drive sleeve 17 advances sufficiently in the distal direction such that it disengages from the axial ratchet teeth engagement 35 with the secondary inner body 12. At this point, the secondary drive sleeve 17 is able to rotate in clockwise direction 47.

Under the action of the secondary dispense spring 20, the drive ratchet 19 and secondary drive sleeve are forced towards the proximal end 6 as shown in FIG. 13. The secondary drive sleeve 17 rotates clockwise 47 and engages with the axial ratchet teeth in the secondary inner body 12, in particular the slanted surface 79 of the secondary inner body 12 such that the secondary drive sleeve 17 moves along a helical path with rotation in clockwise direction. Clockwise rotation of the secondary drive sleeve 17 is transferred to the secondary lead screw 21 via the splined engagement between the secondary drive sleeve 17 and the secondary lead screw 21 with the secondary drive sleeve 17 being rotationally fixed to the secondary lead screw 21. The secondary lead screw 21 is threaded to the secondary drug delivery assembly housing 11 such that rotation of the secondary lead screw 21 moves the secondary lead screw 21 in distal direction. Axial movement of the secondary lead screw 21 and the bearing at the distal end of the secondary lead screw 21 displaces a bung in a cartridge (not shown) attached to the distal end of the device thereby causing a liquid secondary medicament to be dispensed from the cartridge.

Rotation of the secondary drive sleeve 17 continues under the action of the secondary dispense spring 20 until the secondary drive sleeve 17 fully engages with the secondary inner body 12. The angle of rotation of the secondary drive sleeve 17 is determined by the geometry of the ratchet teeth on the secondary inner body 12. In combination with the pitch of the secondary lead screw 21 it determines a fixed axial displacement of the secondary lead screw 21 for a single advancement of the secondary drive sleeve 17.

As the secondary drive sleeve 17 returns towards the proximal end 6, it re-enages with the setting sleeve 23, generating a clockwise rotation of the setting sleeve 23.

The setting sleeve 23 rotates before it engages with the selection sleeve 16. As the setting sleeve 23 rotates, it moves axially, while maintaining its abutment with the lens 52. The selection sleeve 16 is displaced axially by the action of its engagement with the setting sleeve 23, but does not rotate. Displacement of the lens 52 as the trigger is depressed changes the dose number displayed to the user, corresponding to the delivery of the fixed dose. At this point the fixed dose has been dispensed.

Upon completion of the dispensing of the fixed dose, the drive ratchet 19 returns to its proximal position, which removes the splined interface to the clutch 31. The clutch 31 has been displaced axially towards the distal end of the device via its interface with the primary drive sleeve 27 when the trigger was depressed. When the splined interface with the drive ratchet 19 is removed, the clutch 31 is free to rotate. Under the action of the primary dispensing spring, the primary drive sleeve 27 and clutch 31 rotate counterclockwise, thereby causing the number sleeve 30 to travel along its helical path towards its zero unit position.

The primary lead screw 32 rotates via its spline interface to the primary drive sleeve 27. The primary lead screw 32 is threaded to the primary drug delivery assembly housing 10 such that as it rotates counter clockwise it advances towards the distal end 7 of the device. The bearing at the distal end of the primary lead screw 21 is in abutment with a bung with a cartridge attached to the distal end of the primary drug delivery assembly housing. Axial movement of the primary lead screw 32 and the bearing therefore displace the bung, causing a liquid primary medicament to be dispensed from the cartridge.

As liquid primary medicament is dispensed, the motion of the number sleeve 30 causes the dose number displayed to the user by the indices 49 to count down towards zero, in correlation to the dose being dispensed. As the clutch 31 rotates, the radial ribs generate a series of clicks, corresponding to each dose unit, giving audible feedback to the user. Dispensing of the variable dose continues, while the user continues to depress the trigger 24 until the number sleeve 30 returns to the zero dose position. As the number sleeve 30 returns to the zero unit position, the axial abutment between the number sleeve 30 and latch 68 raises the latch 68 towards its proximal position such that the selection shaft 14 is able to rotate and under the action of the selection spring returns to its ‘At Rest’ position. Rotation of the selection shaft 14 allows the remaining components within the fixed dose setting and dispensing mechanisms to return to their ‘At Rest’ positions.

At this point, the variable dose has been dispensed. When the user releases the trigger 24, the action of the integral axial spring which forms part of the primary drug delivery assembly housing 10 and acts on the clutch 31, forces the clutch 31, the primary drive sleeve 27, the trigger 24 and the dial grip 26 to return to their proximal position. The device has therefore been returned to its ‘At Rest’ state.

In the following, dispensing of a dose is explained when a fixed dose in the secondary drug delivery assembly is not set.

As the user depresses the trigger, the trigger moves laterally in distal direction and the dial grip and the primary drive sleeve move axially with the trigger. The dial grip becomes rotationally constrained to the primary drug delivery assembly housing and the flexible ratchet arms on the primary drive sleeve deflect to allow the primary drive sleeve to rotate relative to the dial grip and primary inner body.

As no fixed dose has been set, the lens is positioned in its distal position so that axial displacement of the primary drive sleeve does not generate an abutment which affects the position of the lens. The drive ratchet is located in its proximal position, so that there is no spline engagement between the drive ratchet and the clutch. Therefore, as the primary drive sleeve is displaced axially engaging the axially splined interface with the clutch and causing the clutch to displace axially, the clutch is free to rotate.

Under the action of the primary dispensing spring, the primary drive sleeve and clutch rotate counterclockwise, causing the number sleeve to travel along a helical path towards its zero unit position.

The primary lead screw rotates due to its spline interface to the primary drive sleeve. The primary lead screw is threaded to the primary drug delivery assembly housing, such that as it rotates counterclockwise it advances towards the distal end of the device, thereby displacing a bung in the aforementioned cartridge.

During dispense of a primary medicament in the cartridge by means of the distally moving primary lead screw, the motion of the number sleeve causes the dose number displayed to the user to count down towards zero, in correlation to the dose being dispensed. As the clutch rotates, the radial ribs generate a series of clicks, corresponding to each dose unit, giving audible feedback to the user.

Dispensing of the variable dose continues, while the user continues to depress the trigger, until the number sleeve returns to the zero dose postion. As the number sleeve returns to the zero unit position, the axial abutment between the number sleeve and the latch raises the latch towards its proximal position. As the latch rises, the selection shaft is able to rotate and under the action of the selection spring returns to its ‘At Rest’ position.

Rotation of the selection shaft allows the remaining components within the secondary drug delivery assembly to return to their ‘At Rest’ positions. At this point, the variable dose has been dispensed. When the user releases the trigger, the action of the integral axial spring which forms part of the primary drug delivery assembly housing and acts on the clutch, forces the clutch, the primary drive sleeve, the trigger and the dial grip to return to their proximal position. The device has therefore been returned to its ‘At Rest’ state.

### REFERENCE NUMERALS

1 drug delivery device

2 drug delivery device housing

3 primary drug delivery assembly

4 secondary drug delivery assembly

≡longitudinal axis

6 proximal end

7 distal end

8 variable dose setting and dispensing mechanism (primary dose setting and dispensing mechanism)

9 fixed dose setting and dispensing mechanism (secondary dose setting and dispensing mechanism)

10 primary drug delivery assembly housing

11 secondary drug delivery assembly housing

12 secondary inner body

13 selection element (selection lever)

14 selection shaft

15 selection spring

16 selection sleeve

17 secondary drive sleeve

18 secondary last dose nut

19 ratchet (drive ratchet)

20 secondary dispense spring

21 secondary lead screw

22 bearing

23 setting sleeve

24 trigger

25 primary inner body

26 dial grip

27 primary drive sleeve

28 primary dispense spring

29 spring spool

30 primary dose dial sleeve (number sleeve)

31 clutch

32 primary lead screw

33 longitudinal axis of secondary drug delivery assembly

34 axial ratchet teeth

35 axial ratchet teeth

36 axial ratchet teeth

37 abutment

38 longitudinal axis of the primary drug delivery assembly

39 aperture

40 flexible ratchet arms

41 ratchet teeth in primary inner body

42 ratchet teeth in dial grip

43 splined interface

44 abutment

45 collar

46 storage spool

47 clockwise direction

48 thread

49 indices

50 first number marking

51 second number marking

52 movable element (lens)

53 transparent aperture

54 abutment

55 counterclockwise direction

56 counter abutment surface

57 lens spring

58 axial ratchet teeth

59 lock mechanism (first interlock mechanism)

60 splined interface

61 recessed surface

62 sliding element (two units advance slider)

63 slot

64 profiled abutment surface

65 engagement section

66 counter engagement surface

67 interlock mechanism (latching mechanism)

68 latch

69 distal projection

70 proximal projection

71 profiled boss on selection shaft

72 second axial abutment

73 radial abutment

74 interlock slider

75 slot

76 cam profile

77 projection

78 abutment surface

79 slanted surface

1. Drug delivery device (1) comprising a primary drug delivery assembly (3) for the delivery of a primary medicament with a primary dose setting mechanism (8) and a secondary drug delivery assembly (4) for the delivery of a secondary medicament; and a selection element (13) movable in a first and a second direction and configured such that movement of the selection element (13) in the first direction and in the second direction causes the setting of an increasing dose in the primary dose setting mechanism (8).

2. Drug delivery device according to claim 1, wherein the selection element (13) is moveable between a first, a second and a third position.

3. Drug delivery device according to any of the claims 1 to 2, wherein the selection element (13) is a dose setting element of the secondary drug delivery assembly (4).

4. Drug delivery device according to any of the preceding claims, further comprising a lock mechanism (59) that is configured to lock a primary dose dispensing mechanism (8) against dispensing.

5. Drug delivery device according to claim 4, wherein the lock mechanism (59) is set from an unlocked in a locked state when a dose in a secondary dose setting mechanism (9) of the secondary drug delivery assembly (4) is set.

6. Drug delivery device according to claim 5, wherein the lock mechanism (59) comprises a ratchet (19) and a clutch (31), wherein in the locked state the clutch (31) is rotationally locked to the ratchet (19).

7. Drug delivery device according to claim 6, wherein the clutch (31) is rotationally constrained to a lead screw (32) of the primary drug delivery assembly (3).

8. Drug delivery device according to any of the claims 6 to 7, wherein the ratchet (19) is axially moveable and rotationally constrained.

9. Drug delivery device according to any of the claims 6 to 8, wherein the secondary drug delivery assembly (4) comprises a biasing element (20) to bias the ratchet (19) in proximal direction.

10. Drug delivery device according to claim 9, wherein the biasing element (20) is arranged such that movement of the ratchet (19) into engagement with the clutch (31) compresses the biasing element (20).

11. Drug delivery device according to any of the claims 6 to 10, wherein the secondary drug delivery assembly (4) comprises a secondary drive sleeve (17) configured to move distally during dose setting such that ratchet (19) engages the clutch (31).

12. Drug delivery device according to claim 11, characterized by a coupling mechanism that transfers rotational movement of the selection element (13) from the first into the second position into distal motion of the secondary drive sleeve (17).

13. Drug delivery device according to any of the claims 1 to 12, wherein a transfer element (62) is configured to transfer a setting movement of the selection element (13) to the primary dose setting mechanism (8).

14. Drug delivery device according to any of the claims 2 to 13, wherein a latching means (68) is configured to lock the setting element (13) in the second and/or the third position.

15. Drug delivery device according to any of the claims 2 to 14, comprising a limiting means (74) configured to limit the setting of a dose in the primary drug delivery assembly (8) to a predetermined value when the selection element (13) is in the first position and to allow the setting of higher doses when the selection element (13) is in the second or third position.
